SEQUENCE LISTING (4 sequences)

SEQ ID NO: 1 - protein, 4 aa, Artificial Sequence
  synthetic
  MOD_RES (2)..(2): hyperpolarized alanine
GXEL

SEQ ID NO: 2 - protein, 5 aa, Artificial Sequence
  synthetic
  MOD_RES (3)..(3): hyperpolarized alanine
TGXEL

SEQ ID NO: 3 - protein, 4 aa, Artificial Sequence
  synthetic
  MOD_RES (2)..(2): dehydroalanine
GXEL

SEQ ID NO: 4 - protein, 5 aa, Artificial Sequence
  synthetic
  MOD_RES (3)..(3): dehydroalanine
TGXEL

(19) United States
(12) Patent Application Publication
(10) Pub. No.: US 2018/0230182 A1
(43) Pub. Date: Aug. 16, 2018

(54) HYPERPOLARIZATION OF AMINO ACID RESIDUES FOR PRODUCING HYPERPOLARIZED PEPTIDES

(71) Applicant: Cedars-Sinai Medical Center, Los Angeles, CA (US)

(72) Inventors: Shawn Wagner, Altadena, CA (US); Yi Zhang, Los Angeles, CA (US)

(73) Assignee: Cedars-Sinai Medical Center, Los Angeles, CA (US)

(21) Appl. No.: 15/897,462

(22) Filed: Feb. 15, 2018

Related U.S. Application Data
(60) Provisional application No. 62/459,475, filed on Feb. 15, 2017.

(51) Int. Cl. C07K 5/10; A61K 49/06; C07K 7/06

(52) U.S. Cl. CPC C07K 5/10; C07K 7/06; A61K 49/06

(57) ABSTRACT

The invention relates to the field of Magnetic Resonance Imaging (MRI). More particularly, the invention relates to hyperpolarized peptides for use in MRI.

### CROSS-REFERENCE TO RELATED APPLICATION

This application claims the benefit under 35 U.S.C. § 119(e) of U.S. Provisional Patent Application No. 62/459,475 filed on Feb. 15, 2017, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 14, 2018, is named SequenceListing-065472-000658US00_ST25.txt and is 1,202 bytes in size.

### FIELD OF THE INVENTION

The invention relates to the field of Magnetic Resonance Imaging (MRI). More particularly, the invention relates to hyperpolarized peptides for use in MRI.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Magnetic Resonance Imaging (MRI) is a non-invasive diagnostic tool useful for medical and biological investigations, ex vivo, in vitro and/or in vivo, for example in the investigation of various tissues (e.g., for disease detection or determination of treatment effectiveness). Unfortunately, a drawback which can limit MRI in such investigations is based on the intrinsic low sensitivity of the Nuclear Magnetic Resonance (NMR) spectroscopy on which MRI is based.

For example, to achieve effective contrast between MRI images of different tissue types, it is known to use various contrast agents which affect relaxation times of imaging nuclei (i.e., the nuclei whose MRI signal is used to generate the image) in the tissue which are in proximity to the added contrast agent. MRI signal strength is also dependent on the population difference between the nuclear spin states of the imaging nuclei. This population difference is governed by a Boltzmann distribution and is dependent on temperature and magnetic field.

One proposed solution to the aforementioned drawback of low sensitivity is through the use of hyperpolarized compounds. Over the years efforts have been made to develop various hyperpolarized compounds and MRI methods using these hyperpolarized compounds. The use of hyperpolarized compounds as MRI contrast agents in MRI based investigations has an advantage over other known MRI techniques in that the nuclear polarization to which the MRI signal is proportional is essentially independent of the magnetic field strength of the MRI instrument.

As stated above, various methods and techniques have been developed to prepare hyperpolarized compounds for use in MRI. One polarization method used to increase the population difference between the nuclear spin states of the imaging nuclei has been achieved by using the “Overhauser effect” in which an Electron Spin Resonance (ESR) transition in a paramagnetic species added to the system is coupled to the nuclear spin system of the imaging nuclei. A technique known as Dynamic Nuclear Polarization (DNP), which utilizes the Overhauser effect, has been shown to increase the population difference between the excited and ground nuclear spin state of the imaging nuclei and thereby increase the MRI signal intensity.

Dynamic Nuclear Polarization (DNP), while in principle, may be applied to any type of compound or molecule, possesses several undesirable features which limits its general use. For example, DNP requires relatively long times to perform the polarization step in addition to the need for special cryogenic liquids and hardware required to allow the irradiation of electrons at low temperature. While DNP may be suitable to hyperpolarize some small compounds and small molecules, it is not suitable to hyperpolarize larger compounds and larger molecules, such as biological molecules, peptides, etc., due to the generally poor solubility of these larger compounds and molecules at the low temperatures required for DNP.

Therefore, there is a need for hyperpolarized compounds and hyperpolarized peptides and methods to make such hyperpolarized compounds and hyperpolarized peptides for use in MRI based ex vivo, in vitro and/or in vivo investigations.

### SUMMARY OF THE INVENTION

The following embodiments and aspects thereof are described and illustrated in conjunction with systems, compositions and methods which are meant to be exemplary and illustrative, not limiting in scope.

In various embodiments, the present invention provides a hyperpolarized peptide, comprising: at least one hyperpolarized alanine residue and one or more other amino acid residues. In some embodiments, the one or more other amino acid residues is any natural amino acid or unnatural amino acid. In some embodiments, the one or more other amino acid residues is any natural amino acid. In some embodiments, the at least one hyperpolarized alanine residue comprises a non-hydrogen non-zero nuclear spin nucleus. In some embodiments, the non-hydrogen non-zero nuclear spin nucleus is ¹³C. In some embodiments, the ¹³C is present at a level according to its natural isotopic abundance. In some embodiments, the ¹³C is present at a level greater than its natural isotopic abundance. In some embodiments, one or more hydrogen atoms is replaced with a deuterium atom.

In some embodiments, the non-hydrogen non-zero nuclear spin nucleus is ¹⁵N. In some embodiments, the ¹⁵N is present at a level according to its natural isotopic abundance. In some embodiments, the ¹⁵N is present at a level greater than its natural isotopic abundance.

In various embodiments, the present invention provides a method for preparing a hyperpolarized peptide, comprising: providing a reagent peptide comprising at least one dehydroalanine residue and one or more other amino acid residues; contacting the reagent peptide with molecular hydrogen enriched with para-hydrogen and a hydrogenation catalyst under conditions effective to form a para-hydrogenated peptide; and subjecting the para-hydrogenated peptide to a polarization transfer sequence to obtain the hyperpolarized peptide. In some embodiments, the one or more other amino acid residues is any natural amino acid or unnatural amino acid.

In various embodiments, the present invention provides a method of imaging a subject, comprising: administering a hyperpolarized peptide to the subject; and imaging the subject after administering the hyperpolarized peptide to the subject, wherein the hyperpolarized peptide, comprises at least one hyperpolarized alanine residue and one or more other amino acid residues. In some embodiments, imaging the subject comprises magnetic resonance imaging (MRI) of the subject. In some embodiments, the method further comprises diagnosing the subject as having a disease. In some embodiments, the method further comprises prognosing the subject as being likely to develop a disease. In some embodiments, the method further comprises prognosing the subject as having a higher probability of developing a disease as compared to a control subject, wherein the control subject does not have the disease.

In various embodiments, the present invention provides a method for obtaining one or more magnetic resonance images of a subject, comprising: providing a reagent peptide comprising at least one dehydroalanine residue and one or more other amino acid residues; contacting the reagent peptide with molecular hydrogen enriched with para-hydrogen and a hydrogenation catalyst under conditions effective to form a para-hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; and generating one or more magnetic resonance images of the subject from the detected magnetic resonance signals.

In various embodiments, the present invention provides a method for detecting a disease in a subject, comprising: providing a reagent peptide comprising at least one dehydroalanine residue and one or more other amino acid residues; contacting the reagent peptide with molecular hydrogen enriched with para-hydrogen and a hydrogenation catalyst under conditions effective to form a para-hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals to obtain one or more magnetic resonance images of the subject; comparing the one or more magnetic resonance images of the subject to an image from a reference sample, wherein a change in the one or more magnetic resonance images of the subject relative to the image from the reference sample is indicative of the disease in the subject. In some embodiments, the reference sample is obtained from a control subject, wherein the control subject does not have the disease. In some embodiments, the image from the reference sample is a preexisting image from the subject. In some embodiments, the preexisting image corresponds to a baseline image. In some embodiments, the image from a reference sample is a magnetic resonance image. In some embodiments, the image from a reference sample is a magnetic resonance image of or from the reference sample. In some embodiments, the image from a reference sample is a reference image. In some embodiments, the reference image is a magnetic resonance image. In some embodiments, the reference image is a magnetic resonance image of or from the reference sample.

In various embodiments, the present invention provides a method for treating a subject for a disease, the method comprising: providing a reagent peptide comprising at least one dehydroalanine residue and one or more other amino acid residues; contacting the reagent peptide with molecular hydrogen enriched with para-hydrogen and a hydrogenation catalyst under conditions effective to form a para-hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals to obtain one or more magnetic resonance images of the subject; making an assessment of the subject based on the one or more magnetic resonance images of the subject, wherein the assessment is a detection of the disease; and treating the subject based on the assessment.

In various embodiments, the present invention provides a method for assessing the efficacy of a treatment, comprising: providing a reagent peptide comprising at least one dehydroalanine residue and one or more other amino acid residues; contacting the reagent peptide with molecular hydrogen enriched with para-hydrogen and a hydrogenation catalyst under conditions effective to form a para-hydrogenated peptide; subjecting the para-hydrogenated peptide to a polarization transfer sequence to obtain a hyperpolarized peptide; administering the hyperpolarized peptide to a subject; detecting magnetic resonance signals of the hyperpolarized peptide from the subject; generating one or more magnetic resonance images of the subject from the detected magnetic resonance signals to obtain one or more magnetic resonance images of the subject; making an assessment of the subject based on the one or more magnetic resonance images of the subject, wherein the assessment is a detection of a disease; treating the subject based on the assessment; and comparing the one or more magnetic resonance images of the subject to an image from a reference sample, wherein a change in the one or more magnetic resonance images of the subject relative to the image from the reference sample is indicative of the efficacy of the treatment. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease. In some embodiments, the image from a reference sample is a magnetic resonance image. In some embodiments, the image from a reference sample is a magnetic resonance image of or from the reference sample. In some embodiments, the image from a reference sample is a reference image. In some embodiments, the reference image is a magnetic resonance image. In some embodiments, the reference image is a magnetic resonance image of or from the reference sample.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts in accordance with various embodiments of the invention, a scheme showing the chemical conversion of the dehydroalanine derivative to an alanine derivative followed by Para Hydrogen Induced Polarization (PHIP) of the natural abundance ¹³C alanine carboxyl carbon atom.

FIG. 2A-FIG. 2B depicts in accordance with various embodiments of the invention, polarization experimental maximization by varying the ¹³C-¹H through bond scalar coupling values used to calculate the Goldman RF transfer sequence. FIG. 2A depicts polarization experimental maximization by varying the ¹³C-¹H through bond scalar coupling values used to calculate the Goldman RF transfer sequence. FIG. 2B depicts polarization experimental maximization by varying the ¹³C-¹H through bond scalar coupling values used to calculate the Goldman RF transfer sequence.

FIG. 3 depicts in accordance with various embodiments of the invention, ¹³C spectroscopy obtained on a 9.4T Bruker imaging magnet. The hyperpolarized signal (red) is clearly detectable while 256 averages with a repetition time greater than 5 T₁'s is noisy. Approximation of the intensity puts the hyperpolarization at about 0.2%.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, N.Y. 2006); Smith, March's Advanced Organic Chemistry Reactions, Mechanisms and Structure 7th ed., J. Wiley & Sons (New York, N.Y. 2013), provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ, alkenyl and Cₓ-C_{y}alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Non-limiting examples of Rₐ and R_{b} are each independently hydrogen, alkyl, substituted alkyl, alkenyl, or substituted alkenyl. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ, heteroaryl and Cₓ-C_{y}heteroaryl are typically used where X and Y indicate the number of carbon atoms in the ring system. For example, C₄-C₉ heteroaryl includes heteroaryls that have 4 to 9 carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cycyl are typically used where X and Y indicate the number of carbon atoms in the ring system. For example, C₃-C₈ cyclyl includes cyclyls that have 3 to 8 carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cheterocyclyl and Cₓ-C_{y}heterocyclyl are typically used where X and Y indicate the number of carbon atoms in the ring system. For example, C₄-C₉ heterocyclyl includes heterocyclyls that have 4-9 carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Substituents may be protected as necessary and any of the protecting groups commonly used in the art may be employed. Non-limiting examples of protecting groups may be found, for example, in Greene et al., Protective Groups in Organic Synthesis, 3rd Ed. (New York: Wiley, 1999).

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂— pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂— pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino”-NH-(alkyl)-(cycloalkyl), such as —NHCH₂— cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH3) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

For example, compounds having the present structure except for the replacement of a nitrogen atom by a ¹⁵N-enriched nitrogen are within the scope of the invention.

In various embodiments, compounds of the present invention as disclosed herein may be synthesized using any synthetic method available to one of skill in the art. Non-limiting examples of synthetic methods used to prepare various embodiments of compounds of the present invention are disclosed in the Examples section herein.

As used herein, the terms “treat,” “treatment,” “treating,” or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the disease, disorder, or condition as well as those prone to have the disease, disorder, or condition or those in whom the disease, disorder, or condition is to be prevented.

The term “healthy state” or “normal state” means that the state of the subject (e.g., biological state or health state, etc.) is not abnormal or does not comprise a disease or disorder.

A “healthy subject” or “normal subject” is a subject that does not have a disease or disorder.

Non-limiting examples of treatments or therapeutic treatments include pharmacological or biological therapies and/or interventional surgical treatments.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a disease or disorder. The term “preventative treatment” also means to prevent or slow a subject from obtaining a disease or disorder.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of a disease, delay or slowing of a disease, and amelioration or palliation of symptoms associated with a disease.

The term “disease” refers to an abnormal condition affecting the body of an organism. The term “disorder” refers to a functional abnormality or disturbance.

“Diseases”, and “disease conditions,” as used herein may include, but are in no way limited to any form of cardiovascular conditions, diseases or disorders; or any form of cancer; or any metabolic diseases (e.g., diabetes); or any neurodegenerative diseases (e.g., Alzheimer's); or any enzymatic diseases (e.g., Tay-Sachs).

Cardiovascular diseases are a class of diseases that involve the heart or blood vessels. Non-limiting examples of cardiovascular disease include: coronary artery disease, coronary heart disease, ischemic heart disease (IHD), cardiomyopathy, stroke, hypertensive heart disease, heart failure, pulmonary heart disease, ischemic syndrome, coronary microvascular disease, cardiac dysrhythmias, rheumatic heart disease (RHD), aortic aneurysms, cardiomyopathy, atrial fibrillation, congenital heart disease, endocarditis, inflammatory heart disease, endocarditis, inflammatory cardiomegaly, myocarditis, valvular heart disease, cerebrovascular disease, and peripheral artery disease (PAD).

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), T cell lymphomas, myeloma, myelodysplastic syndrome, skin cancer, brain tumor, breast cancer, colon cancer, rectal cancer, esophageal cancer, anal cancer, cancer of unknown primary site, endocrine cancer, testicular cancer, lung cancer, hepatocellular cancer, gastric cancer, pancreatic cancer, cervical cancer, ovarian cancer, liver cancer, bladder cancer, cancer of the urinary tract, cancer of reproductive organs thyroid cancer, renal cancer, carcinoma, melanoma, head and neck cancer, brain cancer, prostate cancer, including but not limited to androgen-dependent prostate cancer and androgen-independent prostate cancer, and leukemia.

Chemotherapeutic agents are compounds that are known to be of use in chemotherapy for cancer. Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolinodoxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-alpha, Raf, HRas, EGFR (e.g., erlotinib (Tarceva®)) and VEGF-A that reduce cell proliferation and pharmaceutically acceptable salts, acids or derivatives of any of the above or combinations thereof.

“Tumor,” as used herein refers to all neoplastic cell growth and proliferation, whether malignant or benign, and all pre-cancerous and cancerous cells and tissues.

As used herein, the term “administering,” refers to the placement an agent (e.g., a para-hydrogenated compound, a para-hydrogenated peptide, a hyperpolarized compound, or a hyperpolarized peptide) as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

“Sample” is used herein in its broadest sense. The term “biological sample” as used herein denotes a sample taken or isolated from a biological organism. The term “sample” or “biological sample” as used herein denotes a sample taken or isolated from a biological organism. Exemplary biological samples include, but are not limited to, cheek swab; mucus; whole blood, blood, serum; plasma; urine; saliva; semen; lymph; fecal extract; sputum; other body fluid or biofluid; cell sample; and tissue sample etc. The term also includes a mixture of the above-mentioned samples. The term “sample” also includes untreated or pretreated (or pre-processed) biological samples. In some embodiments, a sample can comprise one or more cells from the subject.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, smegma, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device; a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS™) samples can be stored and mailed, and an assay can be performed remotely.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In various embodiments, the subject is human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., a cardiovascular disease) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “reference sample” as used herein means a sample or biological sample that is used as a reference. In some embodiments, a reference sample may be used as a basis for comparison to, with, or against a non-reference sample (e.g., a sample from a subject that is not a reference sample). In some embodiments, the reference sample may be used as a basis for comparison to, with, or against a non-reference sample (e.g., a sample from a subject that is not a reference sample) in order to determine, detect, measure, ascertain, assess, discover, observe, diagnose, treat, and/or prognose (predict) a change, variation, mutation, disease, abnormality, (or lack thereof) in the non-reference sample. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

The term “statistically significant” or “significantly” refers to statistical evidence that there is a difference. It is defined as the probability of making a decision to reject the null hypothesis when the null hypothesis is actually true. The decision is often made using the p-value.

The term “functional” when used in conjunction with “equivalent”, “analog”, “derivative” or “variant” or “fragment” refers to an entity or molecule which possess a biological activity that is substantially similar to a biological activity of the entity or molecule of which it is an equivalent, analog, derivative, variant or fragment thereof.

As used herein, “motion-corrected” means that the raw MRI data that is acquired in the presence of motion (e.g., cardiac and respiratory motion) is retrospectively processed post imaging to remove the motion information which would otherwise appear as image artifacts and confound the interpretation.

As used herein, “registration” or “registered” or “registering” means that multiple images are acquired and the image with least motion is first identified and then the remaining acquisitions are related back to the image with least motion to alter the image features with motion so as to map the images with motion to derive motion-corrected images. The process of mapping back to the image(s) with least motion is referred to as registration herein.

As used herein, the term “amino acid” refers to naturally occurring amino acids (i.e., natural amino acids), unnatural amino acids, and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and 0-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. “Unnatural amino acid” means any amino acid, amino acid derivative, amino acid analog, α-hydroxy acid, or other molecule, other than a natural amino acid.

Peptides are molecules formed by linking at least two amino acids by amide bonds. Amino acids that have been incorporated into peptides are termed “residues” or “amino acid residues” due to the release of either a hydrogen ion from the amine end or a hydroxyl ion from the carboxyl end, or both, as a water molecule is released during formation of each amide bond.

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

“Diagnostic” means identifying the presence or nature of a disease or disorder and includes identifying patients who are at risk of developing a specific disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” While a particular diagnostic method may not provide a definitive diagnosis of a disease or a disorder, it suffices if the method provides a positive indication that aids in diagnosis.

By “at risk of” is intended to mean at increased risk of, compared to a normal subject, or compared to a control group, e.g. a patient population. “Increased risk” or “elevated risk” mean any statistically significant increase in the probability, e.g., that the subject has the disease or disorder. The risk is preferably increased by at least 10%, more preferably at least 20%, and even more preferably at least 50% over the control group with which the comparison is being made.

The terms “detection”, “detecting” and the like, may be used in the context of detecting magnetic resonance signals, detecting magnetic resonance signals of the hyperpolarized peptide, detecting a disease, or detecting a disorder (e.g. when positive assay results are obtained), etc.

The term “phenotype” as used herein comprises the composite of an organism's observable characteristics or traits, such as its morphology, development, biochemical or physiological properties, phenology, behavior, and products of behavior.

The term “diagnosis,” or “dx,” refers to the identification of the nature and cause of a certain phenomenon. As used herein, a diagnosis typically refers to a medical diagnosis, which is the process of determining which disease or disorder explains a symptoms and signs. A diagnostic procedure, often a diagnostic test or assay, can be used to provide a diagnosis. A diagnosis can comprise detecting the presence of a disease or disorder.

The term “prognosis,” or “px,” as used herein refers to predicting the likely outcome of a current standing. For example, a prognosis can include the expected duration and course of a disease or disorder, such as progressive decline or expected recovery.

The term “theranosis,” or “tx” as used herein refers to a diagnosis or prognosis used in the context of a medical treatment. For example, theranostics can include diagnostic testing used for selecting appropriate and optimal therapies (or the inverse) based on the context of genetic content or other molecular or cellular analysis. Theranostics includes pharmacogenomics, personalized and precision medicine.

Pharmaceutical Compositions

In various embodiments, the para-hydrogenated compound, para-hydrogenated peptide, hyperpolarized compound, or hyperpolarized peptide) may be provided as pharmaceutical compositions. In various embodiments, the pharmaceutical compositions according to the invention may be formulated for delivery via any route of administration. “Route of administration” may refer to any administration pathway known in the art, including but not limited to aerosol, nasal, via inhalation, oral, transmucosal, transdermal, parenteral, enteral, topical or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. Methods for these administrations are known to one skilled in the art. In certain embodiments, the pharmaceutical compositions are formulated for intravascular, intravenous, or intraarterial administration. In one embodiment, the pharmaceutical compositions are formulated for intravenous administration as a single bolus.

In some embodiments, the present invention provides a pharmaceutical composition comprising one or more para-hydrogenated compounds. In some embodiments, the present invention provides a pharmaceutical composition comprising one or more para-hydrogenated peptides. In some embodiments, the present invention provides a pharmaceutical composition comprising one or more hyperpolarized compounds. In some embodiments, the present invention provides a pharmaceutical composition comprising one or more hyperpolarized peptides.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its imaging benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical compositions are made following the conventional techniques of pharmacy involving dry milling, mixing, and blending for powder forms; milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in an imaging (e.g., magnetic resonance imaging) effective amount. The precise imaging effective amount is that amount of the composition that will yield the most effective results in terms of imaging the subject. This amount will vary depending upon a variety of factors, including but not limited to the characteristics of the para-hydrogenated compound, para-hydrogenated peptide, hyperpolarized compound, or hyperpolarized peptide (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a stress-imaging effective amount through routine experimentation, for instance, by monitoring image quality and adjusting the dosage accordingly.

Before administration to patients, formulants may be added to the composition. A liquid formulation may be preferred. For example, these formulants may include oils, polymers, vitamins, carbohydrates, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Polymers formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

Many variations and alternative elements have been disclosed in embodiments of the present invention. Still further variations and alternate elements will be apparent to one of skill in the art. Among these variations, without limitation, is the selection of steps, pharmaceutical compositions, administration routes and devices, imaging technologies for the inventive methods, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In pursuit of improved cancer detection, hyperpolarized MRI research has primarily focused on studying metabolic changes supported by hyperpolarized molecules. Based on conversion of the metabolite, this concept builds on changes in chemical structure that result in differences in resonance frequency of the hyperpolarized ¹³C labeled nucleus. However, the capabilities of NMR go much further; for example, physical phenomena can be evaluated by chemical shift, not requiring a chemical change. Leveraging hyperpolarization for signal enhancement opens up new basic science and translational opportunities.

Herein, we present a method for generating a ¹³C hyperpolarizing peptide in a water solution using Para Hydrogen Induced Polarization (PHIP). We demonstrate the feasibility using the hydrogenation conversion of dehydroalanine to alanine in a molecular configuration chemically representative of a peptide. For our model system we are able to achieve 0.2% polarization on a non-deuterated molecule and expect closer to 5% with judicious deuteration around the ¹³C nucleus polarized. Hyperpolarized peptides developed using this method could be used, for example, to evaluate peptide-receptor binding, membrane receptor binding, and/or enzymatic hydrolysis, ex vivo, in vitro or in vivo.

In various embodiments, the present invention provides a method for generating a ¹⁵N hyperpolarizing peptide in a water solution using Para Hydrogen Induced Polarization (PHIP). Hyperpolarized peptides developed using this method could be used, for example, to evaluate peptide-receptor binding, membrane receptor binding, and/or enzymatic hydrolysis, ex vivo, in vitro or in vivo.

In various embodiments, the present invention provides methods to produce a hyperpolarized amino acid, specifically, alanine, or a derivative thereof. In various embodiments, the present invention provides methods to produce a hyperpolarized alanine or a derivative thereof formed in the polarization process using PHIP. In various embodiments, the present invention provides methods to produce a hyperpolarized alanine residue in a peptide, wherein the hyperpolarized alanine residue is formed from a dehydroalanine residue using PHIP.

In various embodiments of the present invention we demonstrate that the carbon-carbon double bond of dehydroalanine is ideally positioned for PHIP to generate a polarized ¹³C carboxy nucleus or a polarized ¹³C carbonyl nucleus with the resulting product being a hyperpolarized alanine residue in various peptides. The dehydroalanine residue can be inserted into a peptide chain using an alanine derivative having a selenium phenol leaving group in combination with various solid state peptide synthesis techniques. The selenium phenol leaving group can be removed using various synthesis techniques to provide a peptide comprising a dehydroalanine residue having a reactive carbon-carbon double bond (e.g., a peptide of Formula IV herein; e.g., a reagent peptide). The carbon-carbon double bond of the dehydroalanine residue is ideally positioned for PHIP to generate a polarized ¹³C carboxy nucleus or a polarized ¹³C carbonyl nucleus with the resulting product being a hyperpolarized peptide comprising a hyperpolarized alanine residue.

In various embodiments of the present invention we demonstrate that the carbon-carbon double bond of dehydroalanine is ideally positioned for PHIP to generate a polarized ¹⁵N amino nucleus or a polarized ¹⁵N amido nucleus or a polarized ¹⁵N amide nucleus with the resulting product being a hyperpolarized alanine residue in various peptides. The dehydroalanine residue can be inserted into a peptide chain using an alanine derivative having a selenium phenol leaving group in combination with various solid state peptide synthesis techniques. The selenium phenol leaving group can be removed using various synthesis techniques to provide a peptide comprising a dehydroalanine residue having a reactive carbon-carbon double bond (e.g., a peptide of Formula IV herein; e.g., a reagent peptide). The carbon-carbon double bond of the dehydroalanine residue is ideally positioned for PHIP to generate a polarized ¹⁵N amino nucleus or a polarized ¹⁵N amido nucleus or a polarized ¹⁵N amide nucleus with the resulting product being a hyperpolarized peptide comprising a hyperpolarized alanine residue.

Para Hydrogen Induced Polarization (PHIP)

Hyperpolarized compounds can be prepared by addition of para-hydrogen to unsaturated substrates by means of a procedure known as Para Hydrogen Induced Polarization (PHIP). The PHIP procedure allows for the formation of populations of the nuclear spin levels that are significantly altered compared to those determined by the Boltzman thermodynamics. An advantage of the PHIP procedure is that is does not require the extremely low temperatures and complex dissolution procedures required by the DNP procedure. The PHIP procedure instead is performed on an unsaturated substrate (e.g., a substrate having a carbon-carbon double bond) by the process of catalytic hydrogenation using hydrogen gas enriched in the para isomer.

The hydrogen molecule (H₂) exists in two isomeric spin forms, ortho-hydrogen (o-H₂), and para-hydrogen (p-H₂). The ortho isomer is symmetric with respect to the exchange of the two protons, is triply degenerate (triplet state), has a spin equal to 1 (S=1), and is NMR active. The para isomer is anti-symmetric, is a singlet state, has a spin equal to 0 (S=0), and is NMR silent.

At room temperature, the two spin forms exist in equilibrium with a 1:3 ratio of para:ortho. At 80 K the two forms exist at 52% (para):48% (ortho) ratio. At 20 K the two forms exist at 99.8% (para):0.2% (ortho). The rate of equilibration between the two isomers is very low. However, in the presence of a suitable catalyst (such as Fe₃O₄, Fe₂O₃, and activated charcoal) an equilibrium mixture is more rapidly obtained. In this manner the para enriched hydrogen can remain stable at room temperature for days to months following removal from the catalyst. Therefore, the para enriched hydrogen has a higher than equilibrium proportion of para-hydrogen, for example, where the proportion of para-hydrogen is more than 25%, more than 30%, more than 45%, more than 50%, more than 60%, more than 70%, more than 80%, more than 90%, more than 95%, more than 99%. In various embodiments, the purity of the para enriched hydrogen is ≥90%, ≥95%, ≥99%, or ≥99%.

Without being bound by theory, when a para-hydrogen (p-H₂) molecule is transferred to a host molecule by means of catalytic hydrogenation, the proton spins remain antiparallel and begin to relax to thermal equilibrium with the normal time constant T₁ near that of a hydrogen atom (e.g., typically about 1 second). However, during relaxation some of the polarization may be transferred to neighboring nuclei by cross-relaxation or other types of coupling. The presence of, for example, a ¹³C nucleus with a suitable substitution pattern close to the relaxing hydrogen may lead to the polarization being conveniently trapped in the slowly relaxing ¹³C nucleus. For example, a ¹³C nucleus in a carbonyl group may have a T₁, relaxation time typically of more than a minute.

Without being bound by theory, the presence of, for example, a ¹⁵N nucleus with a suitable substitution pattern close to the relaxing hydrogen may lead to the polarization being conveniently trapped in the slowly relaxing ¹⁵N nucleus.

For in vivo MRI techniques, the proton signal of a para-hydrogenated compound would overlap with endogenous ¹H signals of the tissue and/or water. However, the near absence of endogenous signal for non-proton nuclei (e.g., ¹³C) results in the practical absence of background noise. This allows for the registration or collection of images with a high signal to noise ratio, where the contrast is only given by the difference in signal intensity between regions reached by the hyperpolarized molecule (e.g., hyperpolarized peptide) and areas in which the hyperpolarized molecule is absent.

Therefore, in various embodiments the present invention provides for the hyperpolarization of ¹³C nuclei in a hyperpolarized peptide, particularly the hyperpolarization of the ¹³C nuclei of the carbonyl carbon of a hyperpolarized alanine residue in a hyperpolarized peptide. In various embodiments the hyperpolarized peptide can be prepared prior to administration of the hyperpolarized peptide to a subject for in vivo MRI investigations. In various embodiments the hyperpolarized peptide is prepared for administration to a sample for in vitro MRI investigations. In various embodiments the hyperpolarized peptide is prepared for administration to a sample for ex vivo MRI investigations. In various embodiments the hyperpolarized peptide is prepared for administration to a sample for ex vivo NMR investigations.

Therefore, in various embodiments the present invention provides for the hyperpolarization of ¹⁵N nuclei in a hyperpolarized peptide, particularly the hyperpolarization of the ¹⁵N nuclei of the amino nitrogen (or ¹⁵N nuclei of the amido nitrogen or ¹⁵N nuclei of the amide nitrogen) of a hyperpolarized alanine residue in a hyperpolarized peptide. In various embodiments the hyperpolarized peptide can be prepared prior to administration of the hyperpolarized peptide to a subject for in vivo MRI investigations. In various embodiments the hyperpolarized peptide is prepared for administration to a sample for in vitro MRI investigations. In various embodiments the hyperpolarized peptide is prepared for administration to a sample for ex vivo MRI investigations. In various embodiments the hyperpolarized peptide is prepared for administration to a sample for ex vivo NMR investigations.

The ¹³C nucleus is a non-hydrogen non-zero nuclear spin nucleus having a nuclear spin=½. The non-hydrogen non-zero nuclear spin nucleus in the hyperpolarized compound or hyperpolarized molecule (e.g., hyperpolarized peptide) may be present in its naturally occurring isotopic abundance. However, where the nucleus is a non-preponderant isotope (e.g., ¹³C) in various embodiments the content of the nucleus (e.g., ¹³C) may be enriched, i.e. present at a higher than naturally occurring level. In various embodiments, the ¹³C enrichment degree is ≥10%, ≥25%, ≥50%, ≥75%, ≥90%, or ≥99%.

The ¹⁵N nucleus is a non-hydrogen non-zero nuclear spin nucleus having a nuclear spin=½. The non-hydrogen non-zero nuclear spin nucleus in the hyperpolarized compound or hyperpolarized molecule (e.g., hyperpolarized peptide) may be present in its naturally occurring isotopic abundance. However, where the nucleus is a non-preponderant isotope (e.g., ¹⁵N) in various embodiments the content of the nucleus (e.g., ¹⁵N) may be enriched, i.e. present at a higher than naturally occurring level. In various embodiments, the ¹⁵N enrichment degree is ≥10%, ≥25%, ≥50%, ≥75%, ≥90%, or ≥99%.

In various embodiments the PHIP procedure is performed using what is generally known as a PHIP instrument. In various embodiments the PHIP instrument is a 1.4 mT PHIP instrument.

In general, the hetero-nuclear hyperpolarization using the PHIP procedure is obtained by transferring the polarization from the hydrogens of the para-hydrogen molecule to the heteronucleus of interest (e.g., ¹³C). In order to use a para-hydrogenated compound (e.g., a peptide comprising an alanine residue comprising one or more para-hydrogen atoms) as, for instance, a ¹³C MRI contrast agent it is necessary that the “anti-phase” signal of the hyperpolarized carbon atom (e.g., carbonyl carbon of an alanine residue comprising one or more para-hydrogen atoms), obtained through polarization transfer from the para-hydrogen to the concerned carbon (e.g., carbonyl carbon of an alanine residue comprising one or more para-hydrogen atoms), is totally converted in an “in phase” signal, useful for imaging acquisition. This step can be performed using an appropriate pulse sequence (e.g., polarization transfer sequence). Non-limiting examples of appropriate pulse sequences (e.g., polarization transfer sequences) are as disclosed, for instance, in Goldman M., Johannesson H., C. R. Phisique 2005, 6, 575, or by applying an appropriated field cycling procedure to the para-hydrogenated product. The latter includes rapidly introducing (non-adiabatically) the hydrogenated sample (e.g., a peptide containing an alanine residue comprising one or more para-hydrogen atoms) into a magnetic screen (e.g., field intensity=0.1 μT), and then slowly removing (adiabatically) the screen to bring the sample to field values corresponding to the Earth's magnetic field (50 μT) (see for example, C.R. Phisique 2004, 5, 315). In various embodiments, the polarization transfer sequence is a Goldman RF transfer sequence. In various embodiments, the polarization transfer sequence is radiofrequency (RF) irradiation. In various embodiments, the polarization transfer sequence is part of the overall PHIP procedure.

In various embodiments, the hetero-nuclear hyperpolarization using the PHIP procedure is obtained by transferring the polarization from the hydrogens of the para-hydrogen molecule to the heteronucleus of interest (e.g., ¹⁵N).

In various embodiments, one or more of the hydrogen atoms of the dehydroalanine residue may be replaced with deuterium atoms, and/or one or more of the other amino acids (or amino acid residues) in the peptide connected to the dehydroalanine residue through a peptide bond or located in proximity to the carbonyl carbon of the dehydroalanine residue may contain deuterium atoms in place of one or more hydrogen atoms in the other amino acids (or amino acid residues). In this way, the polarization transfer to the ¹³C nucleus in the carbonyl carbon of the alanine residue comprising one or more para-hydrogen atoms may be increased.

In various embodiments, one or more of the hydrogen atoms of the dehydroalanine residue may be replaced with deuterium atoms, and/or one or more of the other amino acids (or amino acid residues) in the peptide connected to the dehydroalanine residue through a peptide bond or located in proximity to the carbonyl carbon of the dehydroalanine residue may contain deuterium atoms in place of one or more hydrogen atoms in the other amino acids (or amino acid residues). In this way, the polarization transfer to the ¹⁵N nucleus in the amino nitrogen (or amido nitrogen or amide nitrogen) of the alanine residue comprising one or more para-hydrogen atoms may be increased.

In various embodiments, an alanine amino acid comprising one or more para-hydrogen atoms or a derivative of an alanine amino acid comprising one or more para-hydrogen atoms is subjected to a decoupling sequence. In various embodiments, a peptide, comprising an alanine residue comprising one or more para-hydrogen atoms is subjected to a decoupling sequence. Decoupling can be achieved by irradiating one spin or spin type with the resonant frequency either continuously or with pulse sequences. Non-limiting examples of decoupling sequences (pulse sequences) include MLEV-4, WALTZ-16, etc. Without being bound by theory, it may be advantageous to expose a sample or a subject to a decoupling sequence if the observed nucleus has a low NMR sensitivity (e.g., ¹³C). If the observed nucleus has a low NMR sensitivity, the coupling of the nuclear spins gives a complicated multiplet structure or the coupling information is merely not needed (e.g., homonuclear coupling in heteronuclear correlation spectroscopy). The coupling interactions to the spin that is decoupled are then suppressed, and the multiplet peaks collapse to single lines. The intensity of the signal is the combined intensity of the multiplet lines, which causes a higher signal-to-noise ratio. For example, the spins that are decoupled are the hydrogens (¹H) that are coupled to the ¹³C nucleus. In various embodiments the decoupling sequence is a hydrogen decoupling sequence, wherein the hydrogen (proton) nuclei (¹H) in the sample (e.g., peptide) are irradiated to decouple them from the ¹³C nuclei (e.g., ¹³C nuclei in the carbon atom of the carbonyl group of the alanine residue comprising one or more para-hydrogen atoms in the peptide) being analyzed. This proton (¹H) decoupling can help to increase the intensity of the ¹³C signals. The decoupling sequence may be part of the overall PHIP procedure.

Without being bound by theory, it may be advantageous to expose a sample or a subject to a decoupling sequence if the observed nucleus has a low NMR sensitivity (e.g., ¹⁵N). For example, the spins that are decoupled are the hydrogens (¹H) that are coupled to the ¹⁵N nucleus. In various embodiments the decoupling sequence is a hydrogen decoupling sequence, wherein the hydrogen (proton) nuclei (¹H) in the sample (e.g., peptide) are irradiated to decouple them from the ¹⁵N nuclei (e.g., ¹⁵N nuclei in the nitrogen atom of the amino group (or amido group or amide group) of the alanine residue comprising one or more para-hydrogen atoms in the peptide) being analyzed. This proton (¹H) decoupling can help to increase the intensity of the ¹⁵N signals. The decoupling sequence may be part of the overall PHIP procedure.

In various embodiments, the present invention provides a method of preparing a hyperpolarized peptide, comprising: (a) providing a reagent peptide comprising at least one dehydroalanine residue and one or more other amino acid residues; (b) contacting the reagent peptide with molecular hydrogen enriched with para-hydrogen and a hydrogenation catalyst under conditions effective to hydrogenate the dehydroalanine residue to form a para-hydrogenated peptide, wherein the para-hydrogenated peptide comprises an alanine residue comprising one or more para-hydrogen atoms; (c) subjecting the para-hydrogenated peptide to a decoupling sequence; and (d) subjecting the para-hydrogenated peptide to a polarization transfer sequence.

In various embodiments, the present invention provides a method of preparing a hyperpolarized peptide, comprising: providing a reagent peptide comprising at least one dehydroalanine residue and one or more other amino acid residues; contacting the reagent peptide with molecular hydrogen enriched with para-hydrogen in the presence of a hydrogenation catalyst under conditions effective to hydrogenate the dehydroalanine residue to form a para-hydrogenated peptide, wherein the para-hydrogenated peptide comprises an alanine residue comprising one or more para-hydrogen atoms; subjecting the para-hydrogenated peptide to a decoupling sequence; and subjecting the para-hydrogenated peptide to a polarization transfer sequence.

In some embodiments, the one or more other amino acid residue(s) is not a dehydroalanine residue.

In various embodiments, the present invention provides a method of preparing a hyperpolarized peptide, comprising: (a) providing a reagent peptide comprising at least one dehydroalanine residue and at least one other amino acid residue; (b) contacting the reagent peptide with molecular hydrogen enriched with para-hydrogen and a hydrogenation catalyst under conditions effective to hydrogenate the dehydroalanine residue to form a para-hydrogenated peptide, wherein the para-hydrogenated peptide comprises an alanine residue comprising at least one para-hydrogen atom; (c) subjecting the para-hydrogenated peptide to a decoupling sequence; and (d) subjecting the para-hydrogenated peptide to a polarization transfer sequence.

In various embodiments, the present invention provides a method of preparing a hyperpolarized peptide, comprising: providing a reagent peptide comprising at least one dehydroalanine residue and at least one other amino acid residue; contacting the reagent peptide with molecular hydrogen enriched with para-hydrogen in the presence of a hydrogenation catalyst under conditions effective to hydrogenate the dehydroalanine residue to form a para-hydrogenated peptide, wherein the para-hydrogenated peptide comprises an alanine residue comprising at least one para-hydrogen atom; subjecting the para-hydrogenated peptide to a decoupling sequence; and subjecting the para-hydrogenated peptide to a polarization transfer sequence.

In some embodiments, the at least one other amino acid residue(s) is not a dehydroalanine residue.

Catalytic hydrogenation of the carbon-carbon double bond of the dehydroalanine residue may be performed using any suitable hydrogenation catalyst known in the art. (e.g., for example see K. Goldman et al., Magn Res. Med. 2001, 46 1-5) Hydrogenation catalysts suitable for use in various embodiments of the present invention are known, examples of which are disclosed herein, or if not commercially available, may be prepared by known methods. Conditions suitable for the hydrogenation may be at elevated temperature and/or pressure. During the hydrogenation the entire para-hydrogen enriched hydrogen molecule should be transferred to the carbon-carbon double bond of the dehydroalanine residue present in the compound or peptide. The hydrogenation catalyst used in the hydrogenation may be a heterogeneous catalyst or a homogeneous catalyst. In various embodiments the hydrogenation catalyst is a homogenous transition metal based catalyst. In various embodiments the hydrogenation catalyst is a rhodium complex or iridium complex. In various embodiments the hydrogenation catalyst is a rhodium (I) cationic complex (i.e., Rh(I) cationic complex). In various embodiments the hydrogenation catalyst is a homogenous Rh(I) cationic complex. In various embodiments the hydrogenation catalyst is a homogenous Rh(I) cationic complex comprising a chelating ligand and a diene molecule. In various embodiments the hydrogenation catalyst is a homogenous Rh(I) cationic complex comprising a chelating ligand and a diene molecule, wherein the chelating ligand is a chelating phosphine ligand. Non-limiting examples of chelating phosphine ligands include diphenylphosphine butane (DPPB), diphenylphosphine ethane (DPPE), or (S,S)-1,2-bis(tert-butylmethylphosphino)ethane ((S,S)-t-Bu-BisP*). Non-limiting examples of diene molecules include cyclooctadiene or norbornadiene (nbd). In various embodiments of the present invention, non-limiting examples of the hydrogenation catalyst include rhodium complexes of formula [Rh(diphosphine)diene)]⁺[anion]⁻, where the diphosphine is selected from DPPB (1,4-Bis(diphenylphosphino)butane), DPPE (1,2-Bis(diphenylphosphino)ethane) and derivatives thereof including, for instance, the chiral phosphines such as DINAP (2,2′-Bis(diphenylphosphino)-1,1′-binaftyl), CHIRAPHOS (2,3-diphenylphosphinobutane), DIOP (1,4-Bis(diphenylphosphino)-1,4-bisdeoxy-2,3-O-isopropyliden-L-treitol), and DIP AMP (1,2-Bis[(2-methoxyphenyl)(phenilphosphino)]ethane) In various embodiments of the present invention the diene is selected from 1,5-cyclooctadiene and norbornadiene. In various embodiments of the present invention, the anion can be any anion, but, preferably, tetrafluoroborate or trifluoromethyl sulfonate. In various embodiments of the present invention, the hydrogenation catalyst is [Rh((S,S)-1,2-bis(tert-butylmethylphosphino)ethane) (norbornadiene)]BF₄ (i.e., [Rh((S,S)-t-Bu-BisP*)(nbd)]BF₄).

In various embodiments, the catalytic hydrogenation procedure may be performed in the liquid or gaseous phase, preferably in the absence of materials that would promote relaxation. If the hydrogenation procedure is performed in the liquid phase, the hydrogenation catalyst can be removed by filtration or any other known means. If the hydrogenation procedure is performed in the gas phase then separation of the hydrogenation catalyst may be performed, for example, by passing the hydrogenated compound through a suitable solvent, for example a physiologically suitable solvent, for example water.

In various embodiments, a hydrogenation catalyst used in the catalytic hydrogenation of a dehydroalanine residue of a peptide or compound is removed from the reaction mixture prior to use of the peptide or compound in an in vivo, ex vivo, or in vitro application. In various embodiments, a hydrogenation catalyst used in the catalytic hydrogenation of a dehydroalanine residue of a peptide or compound is removed from the reaction mixture prior to use of the peptide or compound in an in vivo application. In various embodiments, a peptide or compound is purified before being utilized for ex vivo, in vitro or in vivo applications. Without being bound by theory, generally the purification process may be performed using any suitable technique. In some embodiments, the purification process may comprise lyophilization. In various embodiments, purification of a peptide or compound occurs after hydrogenation of the dehydroalanine residue, but before subjecting the peptide to a polarization transfer sequence. In various embodiments, a peptide or compound is subjected to hydrogenation of a dehydroalanine residue and to a polarization transfer sequence before the peptide or compound is purified. In various embodiments, a peptide or compound is not purified and is suitable for use following hydrogenation and polarization of a dehydroalanine residue.

In some embodiments, the hydrogenation reaction may be carried out in an aqueous solution, in an organic solvent, or a combination thereof. In some embodiments, the hydrogenation reaction is carried out in an aqueous solution. In various embodiments if an organic solvent is used during the hydrogenation process, it is removed from the reaction mixture prior to use of the peptide or compound in in vivo applications. In various embodiments, the hydrogenation catalysts comprise ligands having ionic groups and/or polar groups in order to improve the solubility and efficiency of the catalysts in aqueous solution. In various embodiments, non-limiting examples of solvents suitable for use in the present invention may be water (H₂O), deuterated water (D₂O), acetone; organochlorinated solvents, for example, chloroform, dichloromethane, carbon tetrachloride; aromatic solvents, for example, benzene, toluene; ethers, for example, diethylether, diisopropyl ether, butyl ether; aliphatic hydrocarbons, for example, pentane, hexane, heptane, cyclohexane, ethyl acetate; alcohols, for example, methanol, ethanol, butanol; or any combinations thereof. Solvents may be deuterated or non-deuterated.

According to the present invention, and unless otherwise indicated, the term “aqueous solution” or “suitable aqueous solution”, herein used interchangeably, refers to a sterile water or saline solution, optionally properly buffered, in any case physiologically tolerable and usable in in vivo, ex vivo, or in vitro diagnostic imaging applications (e.g., MRI). Furthermore, an aqueous solution as defined above, further including a suitable amount of a properly selected reagent capable of promoting the rapid and selective conversion of the hyperpolarized molecule (e.g., hyperpolarized compound or hyperpolarized peptide) into a water soluble derivative and to generate, as a result, a physiologically acceptable aqueous solution of the same, suitable for use in in vivo, ex vivo, or in vitro diagnostic imaging (e.g., MRI) without further purification.

In various embodiments, the present invention provides an aqueous solution comprising water, a physiological saline solution, an aqueous solution containing the minimum amount of a base, e.g. NaOH, or of an acid such as citric acid or acetic acid, to give a water soluble and physiologically compatible derivative of a suitable para-hydrogenated substrate or hyperpolarized substrate, for instance in the form of a physiologically acceptable salt, thereof, e.g. of an acid or amine.

Any known MRI instrument and/or MRI technique may be used in the various embodiments and/or methods of the present invention for imaging a subject and/or a sample (e.g., magnetic resonance imaging a subject and/or a sample). In various embodiments, a magnetic resonance image (e.g., a magnetic resonance image of a subject and/or a sample) is obtained using any known MRI instrument and/or MRI technique. In various embodiments, any known MRI instrument and/or MRI technique may be used for imaging (e.g., magnetic resonance imaging) a subject and/or a sample to obtain a magnetic resonance image of the subject and/or the sample. In some embodiments, the sample is a reference sample. In some embodiments, the sample is a non-reference sample.

In various embodiments, a hyperpolarized peptide or compound of the present invention may be used in in vitro, ex vivo, and/or in vivo magnetic resonance imaging (MRI) of a subject and/or sample. In various embodiments, a hyperpolarized peptide or compound of the present invention may be used in in vitro, ex vivo, and/or in vivo magnetic resonance imaging (MRI) of a subject and/or sample for diagnosis and/or detection of disease in the subject and/or sample. In various embodiments, a hyperpolarized peptide or compound of the present invention may be used in in vitro, ex vivo, and/or in vivo magnetic resonance imaging (MRI) of a subject and/or sample for a diagnostic assessment of a physiological process and/or physiological parameters in the subject and/or sample.

Hyperpolarized Peptides

In various embodiments, the present invention provides a hyperpolarized peptide, comprising: at least one hyperpolarized alanine residue and one or more other amino acid residues. In some embodiments, the one or more other amino acid residues is any natural amino acid or unnatural amino acid. In some embodiments, the one or more other amino acid residues is any natural amino acid. In some embodiments, the at least one hyperpolarized alanine residue comprises a non-hydrogen non-zero nuclear spin nucleus. In some embodiments, the non-hydrogen non-zero nuclear spin nucleus is ¹³C. In some embodiments, the ¹³C is present at an amount according to its natural isotopic abundance. In some embodiments, the ¹³C is present at a level greater than its natural isotopic abundance. In some embodiments, the carbonyl carbon of the hyperpolarized alanine residue comprises an isotope ¹³C, wherein the isotope ¹³C is present in an amount according to its natural isotopic abundance or in an amount greater than its natural isotopic abundance. In some embodiments, the hyperpolarized peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, the hyperpolarized peptide has been exposed to or subjected to a polarization transfer sequence. In various embodiments, one or more of the hydrogen atoms of the hyperpolarized peptide is replaced with a deuterium atom.

In some embodiments, the one or more other amino acid residues are independently selected from any amino acid. In some embodiments, the one or more other amino acid residues are independently selected from any natural amino acid and any unnatural amino acid. In some embodiments, the one or more other amino acid residues are independently selected from any natural amino acid.

In some embodiments, the one or more other amino acid residues are independently selected from any amino acid residue. In some embodiments, the one or more other amino acid residues are independently selected from any natural amino acid residue and any unnatural amino acid residue. In some embodiments, the one or more other amino acid residues are independently selected from any natural amino acid residue.

In various embodiments, the present invention provides a hyperpolarized peptide, comprising: at least one hyperpolarized alanine residue; and at least one other amino acid residue. In some embodiments, the at least one other amino acid residue is independently selected from any amino acid residue. In some embodiments, the at least one other amino acid residue is independently selected from any natural amino acid residue and any unnatural amino acid residue. In some embodiments, the at least one other amino acid residue is independently selected from any natural amino acid residue. In some embodiments, the at least one other amino acid residue is not a hyperpolarized alanine residue. In some embodiments, the at least one other amino acid residue is independently selected from any amino acid. In some embodiments, the at least one other amino acid residue is independently selected from any natural amino acid and any unnatural amino acid. In some embodiments, the at least one other amino acid residue is independently selected from any natural amino acid. In some embodiments, the amino acids are amphoteric compounds and may exist in different ionic forms including without limitation a neutral form, a zwitterion form, a salt form, a protected form, or a combination thereof.

In various embodiments, the present invention provides a hyperpolarized peptide, comprising: at least one hyperpolarized alanine residue; and at least one amino acid residue. In some embodiments, the at least one amino acid residue is independently selected from any amino acid residue. In some embodiments, the at least one amino acid residue is independently selected from any natural amino acid residue and any unnatural amino acid residue. In some embodiments, the at least one amino acid residue is independently selected from any natural amino acid residue. In some embodiments, the at least one amino acid residue is not a hyperpolarized alanine residue. In some embodiments, the at least one amino acid residue is independently selected from any amino acid. In some embodiments, the at least one amino acid residue is independently selected from any natural amino acid and any unnatural amino acid. In some embodiments, the at least one amino acid residue is independently selected from any natural amino acid. In some embodiments, the amino acids are amphoteric compounds and may exist in different ionic forms including without limitation a neutral form, a zwitterion form, a salt form, a protected form, or a combination thereof.

In some embodiments, the non-hydrogen non-zero nuclear spin nucleus is ¹⁵N. In some embodiments, the ¹⁵N is present at an amount according to its natural isotopic abundance. In some embodiments, the ¹⁵N is present at a level greater than its natural isotopic abundance. In some embodiments, the amino nitrogen (or amido nitrogen or amide nitrogen) of the hyperpolarized alanine residue comprises an isotope ¹⁵N, wherein the isotope ¹⁵N is present in an amount according to its natural isotopic abundance or in an amount greater than its natural isotopic abundance.

In some embodiments, the one or more other amino acid residues is not a hyperpolarized alanine residue.

In some embodiments, the hyperpolarized peptide is any enantiomer, diastereomer, racemic mixture, enantiomerically enriched mixture, or enantiomerically pure form.

In various embodiments, the present invention provides a hyperpolarized peptide, comprising: at least one hyperpolarized alanine residue; and two or more other amino acid residues. In various embodiments, the present invention provides a hyperpolarized peptide, comprising: at least one hyperpolarized alanine residue and three or more other amino acid residues. In various embodiments, the present invention provides a hyperpolarized peptide, comprising: at least one hyperpolarized alanine residue and four or more other amino acid residues. In various embodiments, the present invention provides a hyperpolarized peptide, comprising: at least one hyperpolarized alanine residue and five or more other amino acid residues. In various embodiments, the present invention provides a hyperpolarized peptide, comprising: at least one hyperpolarized alanine residue and six or more other amino acid residues. In various embodiments, the present invention provides a hyperpolarized peptide, comprising: at least one hyperpolarized alanine residue and seven or more other amino acid residues. In various embodiments, the present invention provides a hyperpolarized peptide, comprising: at least one hyperpolarized alanine residue and eight or more other amino acid residues. In various embodiments, the present invention provides a hyperpolarized peptide, comprising: at least one hyperpolarized alanine residue and nine or more other amino acid residues. In various embodiments, the present invention provides a hyperpolarized peptide, comprising: at least one hyperpolarized alanine residue and ten or more other amino acid residues.

In various embodiments, the present invention provides a hyperpolarized peptide, comprising: at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hyperpolarized alanine residues; and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more other amino acid residues.

In various embodiments, the present invention provides a hyperpolarized peptide, comprising: at least one hyperpolarized alanine residue; and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more other amino acid residues.

In various embodiments, the present invention provides a hyperpolarized peptide, comprising: at least one hyperpolarized alanine residue; and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 other amino acid residue(s).

In various embodiments, the present invention provides a hyperpolarized peptide, comprising: at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hyperpolarized alanine residue(s); and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 other amino acid residue(s).

In various embodiments, the present invention provides a hyperpolarized peptide, comprising: at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hyperpolarized alanine residue(s); and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residue(s).

In various embodiments, the present invention provides a hyperpolarized peptide, comprising: at least one hyperpolarized alanine residue and one or more other amino acid residues, wherein the hyperpolarized alanine residue is of Formula I:

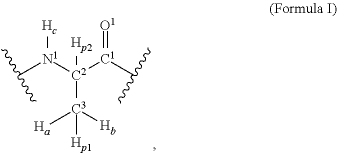

wherein, Hₚ₁ and Hₚ₂ are hydrogen atoms derived from molecular hydrogen enriched in para-hydrogen, or Hₚ₁ and Hₚ₂ are independently an ortho-hydrogen atom or a para-hydrogen atom; C¹ is a carbon atom; C² is a carbon atom; C³ is a carbon atom; N¹ is a nitrogen atom; O¹ is an oxygen atom; Hₐ is a hydrogen atom or a deuterium atom; H_{b} is a hydrogen atom or a deuterium atom; and H_{c} is a hydrogen atom or a deuterium atom. In some embodiments, the one or more other amino acid residues is any natural amino acid or unnatural amino acid. In some embodiments, the one or more other amino acid residues is any natural amino acid. In some embodiments, the carbon atom C¹ comprises an isotope ¹³C, wherein the isotope ¹³C is present in an amount according to its natural isotopic abundance or in an amount greater than its natural isotopic abundance. In some embodiments, Hₐ, H_{b}, or H_{c} or any other hydrogen atoms may independently be a deuterium atom, with the proviso that Hₚ₁ is not a deuterium atom or a tritium atom, and Hₚ₂ is not a deuterium atom or a tritium atom. In some embodiments, the hyperpolarized peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the hyperpolarized peptide has been exposed to or subjected to a polarization transfer sequence.

In some embodiments, the hyperpolarized peptide has not been exposed to or subjected to a decoupling sequence. In some embodiments, the hyperpolarized peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, the hyperpolarized peptide of Formula I is any enantiomer, diastereomer, racemic mixture, enantiomerically enriched mixture, or enantiomerically pure form.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

In various embodiments, the present invention provides a hyperpolarized peptide, of Formula II:

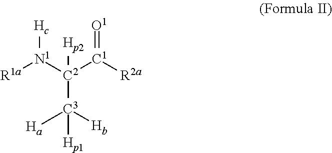

wherein, Hₚ₁ and Hₚ₂ are hydrogen atoms derived from molecular hydrogen enriched in para-hydrogen, or Hₚ₁, and Hₚ₂ are independently an ortho-hydrogen atom or a para-hydrogen atom; C¹ is a carbon atom; C² is a carbon atom; C³ is a carbon atom; N¹ is a nitrogen atom; O¹ is an oxygen atom; Hₐ is a hydrogen atom or a deuterium atom; H_{b} is a hydrogen atom or a deuterium atom; H_{c} is a hydrogen atom or a deuterium atom; R^{1a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, —C(O)—R^{3a}, and one or more other amino acid residues, wherein the one or more other amino acid residues is any natural amino acid or any unnatural amino acid, wherein R^{3a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; R^{2a} is selected from —OR^{4a} and one or more other amino acid residues, wherein the one or more other amino acid residues is any natural amino acid or any unnatural amino acid, wherein R^{4a} is absent or selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; with the proviso that one of R^{1a} or R^{2a} is one or more other amino acid residues.

In some embodiments, R^{1a} is one or more amino acid residues. In some embodiments, the one or more amino acid residues are independently selected from any amino acid. In some embodiments, the one or more amino acid residues are independently selected from any amino acid residue.

In some embodiments, R^{1a} is one or more other amino acid residues. In some embodiments, the one or more other amino acid residues are independently selected from any amino acid. In some embodiments, the one or more other amino acid residues are independently selected from any amino acid residue.

In some embodiments, R^{2a} is one or more amino acid residues. In some embodiments, the one or more amino acid residues are independently selected from any amino acid. In some embodiments, the one or more amino acid residues are independently selected from any amino acid residue.

In some embodiments, R^{2a} is one or more other amino acid residues. In some embodiments, the one or more other amino acid residues are independently selected from any amino acid. In some embodiments, the one or more other amino acid residues are independently selected from any amino acid residue.

In some embodiments, R^{2a} is selected from N(R^{5a})(R^{5b}), wherein R^{5a} and R^{5b} are independently selected from hydrogen, deuterium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, R^{5a} and R^{5b} are optionally linked. In some embodiments, R^{2a} is selected from N(R^{5a})(R^{5b}), and R^{1a} is one or more other amino acid residues. In some embodiments, N(R^{5a})(R^{5b}) is not an amino acid residue. In some embodiments, N(R^{5a})(R^{5b}) is an amino acid residue. In some embodiments, the amino acid residue is from any natural amino acid or any unnatural amino acid. In some embodiments, the amino acid residue is from any amino acid.

In some embodiments, the one or more amino acids is at least one amino acid. In some embodiments, the one or more other amino acids is at least one other amino acid. In some embodiments, the one or more amino acid residues is at least one amino acid residue. In some embodiments, the one or more other amino acid residues is at least one other amino acid residue.

In some embodiments, the hyperpolarized peptide of Formula II is any enantiomer, diastereomer, racemic mixture, enantiomerically enriched mixture, or enantiomerically pure form.

In some embodiments, the carbon atom C¹ comprises an isotope ¹³C, wherein the isotope ¹³C is present in an amount according to its natural isotopic abundance or in an amount greater than its natural isotopic abundance. In some embodiments, Hₐ, H_{b}, or H_{c} or any other hydrogen atoms may independently be a deuterium atom, with the proviso that Hₚ₁ is not a deuterium atom or a tritium atom, and Hₚ₂ is not a deuterium atom or a tritium atom. In some embodiments, the hyperpolarized peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the hyperpolarized peptide has been exposed to or subjected to a polarization transfer sequence.

In some embodiments, the hyperpolarized peptide has not been exposed to or subjected to a decoupling sequence. In some embodiments, the hyperpolarized peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In various embodiments, the natural amino acids may be represented by their name, three letter code, or one letter code as known in the art. In some embodiments, the natural amino acid is selected from alanine (Ala) (A), arginine (Arg) (R), asparagine (Asn) (N), aspartic acid (Asp) (D), cysteine (Cys) (C), glutamic acid (Glu) (E), glutamine (Gln) (Q), glycine (Gly) (G), histidine (His) (H), isoleucine (Ile) (I), leucine (Leu) (L), lysine (Lys) (K), methionine (Met) (M), phenylalanine (Phe) (F), proline (Pro) (P), serine (Ser) (S), threonine (Thr) (T), tryptophan (Trp) (W), tyrosine (Tyr) (Y), and valine (Val) (V).

In various embodiments, the amino acid is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the other amino acid(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the chiral amino acid is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from an Ala, Ile, Leu, Met, Val, Phe, Trp, Tyr, Asn, Cys, Gin, Ser, Thr, Asp, Glu, Arg, His, Lys, and Pro amino acid residue.

In various embodiments, the non-chiral amino acid is Gly. In various embodiments, the non-chiral amino acid residue is Gly. In various embodiments, the non-chiral amino acid residue is a Gly amino acid residue.

In some embodiments, one or more of the amino acids (or amino acid residues) are D-amino acids (or D-amino acid residues). In various embodiments, one or more D-amino acids are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gln, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro amino acid residues.

In various embodiments, the other amino acid residues are D-amino acids. In various embodiments, the other amino acid residues are D-amino acid residues.

In some embodiments, one or more of the amino acids (or amino acid residues) are L-amino acids (or L-amino acid residues). In some embodiments, one or more L-amino acids are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro amino acid residues.

In various embodiments, the other amino acid residues are L-amino acids. In various embodiments, the other amino acid residues are L-amino acid residues.

Non-limiting examples of hyperpolarized peptide of Formula II include:

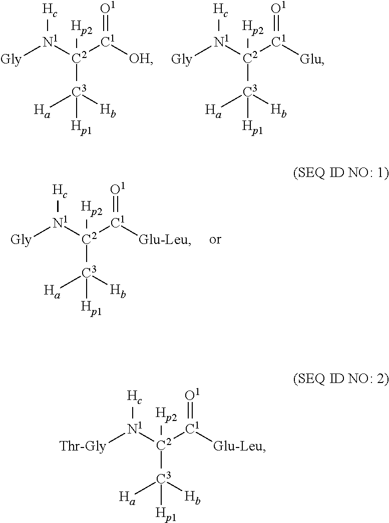

wherein Hₚ₁, Hₚ₂, C¹, C², C³, N¹, O¹, Hₐ, H_{b}, and H_{c} are as defined for Formula I or Formula II. In some embodiments, a non-limiting example of a hyperpolarized peptide of Formula II is Gly-Xaa1-Glu-Leu, wherein Xaa1 is equal to Formula I. In some embodiments, a non-limiting example of a hyperpolarized peptide of Formula II is Thr-Gly-Xaa1-Glu-Leu, wherein Xaa1 is equal to Formula I.

A non-limiting example of hyperpolarized peptide of Formula II is:

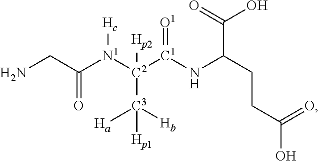

wherein Hₚ₁, Hₚ₂, C¹, C², C³, N¹, O¹, Hₐ, H_{b}, and H_{c} are as defined for Formula I or Formula II.

In various embodiments, the present invention provides a method of preparing a hyperpolarized peptide, comprising: (a) providing a reagent peptide comprising at least one dehydroalanine residue and one or more other amino acid residues; (b) contacting the reagent peptide with molecular hydrogen enriched in para-hydrogen and a hydrogenation catalyst under conditions effective to hydrogenate the dehydroalanine residue to form a para-hydrogenated peptide, wherein the para-hydrogenated peptide comprises an alanine residue comprising one or more para-hydrogen atoms; and (c) subjecting the para-hydrogenated peptide to a polarization transfer sequence. In some embodiments, the method further comprises subjecting the peptide to a decoupling sequence. In some embodiments, the one or more other amino acid residues is any natural amino acid or unnatural amino acid. In some embodiments, the one or more other amino acid residues is any natural amino acid. In some embodiments, the alanine residue comprises a non-hydrogen non-zero nuclear spin nucleus. In some embodiments, the non-hydrogen non-zero nuclear spin nucleus is ¹³C. In some embodiments, the ¹³C is present at an amount according to its natural isotopic abundance. In some embodiments, the ¹³C is present at a level greater than its natural isotopic abundance. In some embodiments, the carbonyl carbon of the alanine residue comprises an isotope ¹³C, wherein the isotope ¹³C is present in an amount according to its natural isotopic abundance or in an amount greater than its natural isotopic abundance. In some embodiments, the hyperpolarized peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, the hyperpolarized peptide comprises a hyperpolarized alanine residue. In some embodiments, the dehydroalanine residue is of Formula III:

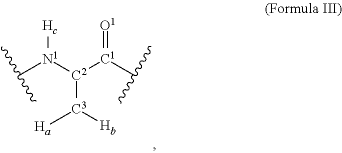

wherein, C¹ is a carbon atom; C² is a carbon atom; C³ is a carbon atom; N¹ is a nitrogen atom; O¹ is an oxygen atom; Hₐ is a hydrogen atom or a deuterium atom; H_{b} is a hydrogen atom or a deuterium atom; and H_{c} is a hydrogen atom or a deuterium atom. In some embodiments, the carbon atom C¹ comprises an isotope ¹³C, wherein the isotope ¹³C is present in an amount according to its natural isotopic abundance or in an amount greater than its natural isotopic abundance. In some embodiments, Hₐ, H_{b}, or H_{c} may independently be deuterium atoms.

In some embodiments, the non-hydrogen non-zero nuclear spin nucleus is ¹⁵N. In some embodiments, the ¹⁵N is present at an amount according to its natural isotopic abundance. In some embodiments, the ¹⁵N is present at a level greater than its natural isotopic abundance. In some embodiments, the amino nitrogen (or amido nitrogen or amide nitrogen) of the alanine residue comprises an isotope ¹⁵N, wherein the isotope ¹⁵N is present in an amount according to its natural isotopic abundance or in an amount greater than its natural isotopic abundance.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, the one or more other amino acid residue(s) is not a dehydroalanine residue.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms.

In some embodiments, the peptide comprising at least one dehydroalanine residue and one or more other amino acid residues is of Formula IV,

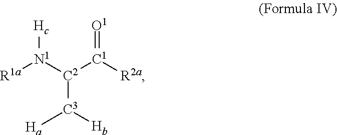

wherein, C¹ is a carbon atom; C² is a carbon atom; C³ is a carbon atom; N¹ is a nitrogen atom; O¹ is an oxygen atom; Hₐ is a hydrogen atom or a deuterium atom; H_{b} is a hydrogen atom or a deuterium atom; H_{c} is a hydrogen atom or a deuterium atom; R^{1a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, —C(O)—R^{3a}, and one or more other amino acid residues, wherein the one or more other amino acid residues is any natural amino acid or any unnatural amino acid, wherein R^{3a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; R^{2a} is selected from —OR^{4a} and one or more other amino acid residues, wherein the one or more other amino acid residues is any natural amino acid or any unnatural amino acid, wherein R^{4a} is absent or selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; with the proviso that one of R^{1a} or R^{2a} is one or more other amino acid residues. In some embodiments the peptide of Formula IV is a reagent peptide.

In some embodiments, R^{1a} is one or more amino acid residues. In some embodiments, the one or more amino acid residues are independently selected from any amino acid. In some embodiments, the one or more amino acid residues are independently selected from any amino acid residue.

In some embodiments, R^{1a} is one or more other amino acid residues. In some embodiments, the one or more other amino acid residues are independently selected from any amino acid. In some embodiments, the one or more other amino acid residues are independently selected from any amino acid residue.

In some embodiments, R^{2a} is one or more amino acid residues. In some embodiments, the one or more amino acid residues are independently selected from any amino acid. In some embodiments, the one or more amino acid residues are independently selected from any amino acid residue.

In some embodiments, R^{2a} is one or more other amino acid residues. In some embodiments, the one or more other amino acid residues are independently selected from any amino acid. In some embodiments, the one or more other amino acid residues are independently selected from any amino acid residue.

In some embodiments, the one or more other amino acid residue(s) is not a dehydroalanine residue.

In some embodiments, R^{2a} is selected from N(R^{5a})(R^{5b}), wherein R^{5a} and R^{5b} are independently selected from hydrogen, deuterium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, R^{5a} and R^{5b} are optionally linked. In some embodiments, R^{2a} is selected from N(R^{5a})(R^{5b}), and R^{1a} is one or more other amino acid residues. In some embodiments, N(R^{5a})(R^{5b}) is not an amino acid residue. In some embodiments, N(R^{5a})(R^{5b}) is an amino acid residue. In some embodiments, the amino acid residue is from any natural amino acid or any unnatural amino acid. In some embodiments, the amino acid residue is from any amino acid.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

In various embodiments, the present invention provides a para-hydrogenated peptide, comprising: at least one alanine residue comprising one or more para-hydrogen atoms and one or more other amino acid residues. In some embodiments, the one or more other amino acid residues is any natural amino acid or unnatural amino acid. In some embodiments, the one or more other amino acid residues is any natural amino acid. In some embodiments, the at least one alanine residue comprising one or more para-hydrogen atoms comprises a non-hydrogen non-zero nuclear spin nucleus. In some embodiments, the non-hydrogen non-zero nuclear spin nucleus is ¹³C. In some embodiments, the ¹³C is present at an amount according to its natural isotopic abundance. In some embodiments, the ¹³C is present at a level greater than its natural isotopic abundance. In some embodiments, the carbonyl carbon of the alanine residue comprises an isotope ¹³C, wherein the isotope ¹³C is present in an amount according to its natural isotopic abundance or in an amount greater than its natural isotopic abundance. In some embodiments, the para-hydrogenated peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In various embodiments, one or more of the hydrogen atoms of the para-hydrogenated peptide is replaced with a deuterium atom.

In some embodiments, the non-hydrogen non-zero nuclear spin nucleus is ¹⁵N. In some embodiments, the ¹⁵N is present at an amount according to its natural isotopic abundance. In some embodiments, the ¹⁵N is present at a level greater than its natural isotopic abundance. In some embodiments, the amino nitrogen (or amido nitrogen or amide nitrogen) of the alanine residue comprises an isotope ¹⁵N, wherein the isotope ¹⁵N is present in an amount according to its natural isotopic abundance or in an amount greater than its natural isotopic abundance.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, the para-hydrogenated peptide is a hyperpolarizable peptide. In some embodiments, the one or more other amino acid residue(s) is not an alanine residue comprising one or more para-hydrogen atoms.

In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

Formula Ia

In various embodiments, the present invention provides a para-hydrogenated peptide, comprising: at least one alanine residue comprising one or more para-hydrogen atoms and one or more other amino acid residues, wherein the alanine residue comprising one or more para-hydrogen atoms is of Formula Ia:

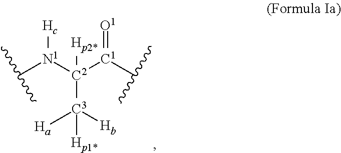

wherein, H_{p1*} and H_{p2*} are hydrogen atoms derived from molecular hydrogen enriched in para-hydrogen, or H_{p1*} and H_{p2*} are independently an ortho-hydrogen atom or a para-hydrogen atom; C¹ is a carbon atom; C² is a carbon atom; C³ is a carbon atom; N¹ is a nitrogen atom; O¹ is an oxygen atom; Hₐ is a hydrogen atom or a deuterium atom; H_{b} is a hydrogen atom or a deuterium atom; and H_{c} is a hydrogen atom or a deuterium atom. In some embodiments, the one or more other amino acid residues is any natural amino acid or unnatural amino acid. In some embodiments, the one or more other amino acid residues is any natural amino acid. In some embodiments, the carbon atom C¹ comprises an isotope ¹³C, wherein the isotope ¹³C is present in an amount according to its natural isotopic abundance or in an amount greater than its natural isotopic abundance. In some embodiments, Hₐ, H_{b}, or H_{c} or any other hydrogen atoms may independently be a deuterium atom, with the proviso that H_{p1*} is not a deuterium atom or a tritium atom, and H_{p2*} is not a deuterium atom or a tritium atom. In some embodiments, the para-hydrogenated peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the para-hydrogenated peptide has not been exposed to or subjected to a polarization transfer sequence.

In some embodiments, the para-hydrogenated peptide has not been exposed to or subjected to a decoupling sequence. In some embodiments, the para-hydrogenated peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, the para-hydrogenated peptide is a hyperpolarizable peptide. In some embodiments, the one or more other amino acid residue(s) is not an alanine residue comprising one or more para-hydrogen atoms.

In some embodiments, the para-hydrogenated peptide is any enantiomer, diastereomer, racemic mixture, enantiomerically enriched mixture, or enantiomerically pure form. In some embodiments, the para-hydrogenated peptide of Formula Ia is any enantiomer, diastereomer, racemic mixture, enantiomerically enriched mixture, or enantiomerically pure form.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

Formula IIa

In various embodiments, the present invention provides a para-hydrogenated peptide of Formula IIa:

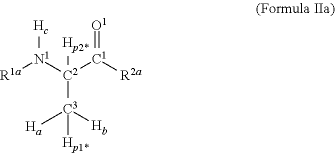

wherein, H_{p1*} and H_{p2*} are hydrogen atoms derived from molecular hydrogen enriched in para-hydrogen, or H_{p1*} and H_{p2*} are independently an ortho-hydrogen atom or a para-hydrogen atom; C¹ is a carbon atom; C² is a carbon atom; C³ is a carbon atom; N¹ is a nitrogen atom; O¹ is an oxygen atom; Hₐ is a hydrogen atom or a deuterium atom; H_{b} is a hydrogen atom or a deuterium atom; H_{c} is a hydrogen atom or a deuterium atom; R^{1a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, —C(O)—R^{3a}, and one or more other amino acid residues, wherein the one or more other amino acid residues is any natural amino acid or any unnatural amino acid, wherein R^{3a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; R^{2a} is selected from —OR^{4a} and one or more other amino acid residues, wherein the one or more other amino acid residues is any natural amino acid or any unnatural amino acid, wherein R^{4a} is absent or selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; with the proviso that one of R^{1a} or R^{2a} is one or more other amino acid residues.

In some embodiments, R^{2a} is selected from N(R^{5a})(R^{5b}), wherein R^{5a} and R^{5b} are independently selected from hydrogen, deuterium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, R^{5a} and R^{5b} are optionally linked. In some embodiments, R^{2a} is selected from N(R^{5a})(R^{5b}), and R^{1a} is one or more other amino acid residues. In some embodiments, N(R^{5a})(R^{5b}) is not an amino acid residue. In some embodiments, N(R^{5a})(R^{5b}) is an amino acid residue. In some embodiments, the amino acid residue is from any natural amino acid or any unnatural amino acid. In some embodiments, the amino acid residue is from any amino acid.

In some embodiments, R^{1a} is one or more amino acid residues. In some embodiments, the one or more amino acid residues are independently selected from any amino acid. In some embodiments, the one or more amino acid residues are independently selected from any amino acid residue.

In some embodiments, R^{1a} is one or more other amino acid residues. In some embodiments, the one or more other amino acid residues are independently selected from any amino acid. In some embodiments, the one or more other amino acid residues are independently selected from any amino acid residue.

In some embodiments, R^{2a} is one or more amino acid residues. In some embodiments, the one or more amino acid residues are independently selected from any amino acid. In some embodiments, the one or more amino acid residues are independently selected from any amino acid residue.

In some embodiments, R^{2a} is one or more other amino acid residues. In some embodiments, the one or more other amino acid residues are independently selected from any amino acid. In some embodiments, the one or more other amino acid residues are independently selected from any amino acid residue.

In some embodiments, the one or more other amino acid residues is at least one other amino acid. In some embodiments, the one or more other amino acid residues is at least one other amino acid residue.

In some embodiments, the para-hydrogenated peptide of Formula IIa is any enantiomer, diastereomer, racemic mixture, enantiomerically enriched mixture, or enantiomerically pure form.

In some embodiments, the para-hydrogenated peptide is a hyperpolarizable peptide. In some embodiments, the one or more other amino acid residue(s) is not an alanine residue comprising one or more para-hydrogen atoms. In some embodiments, the one or more other amino acid residue(s) is optionally an alanine residue comprising one or more para-hydrogen atoms. In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms. In some embodiments, the alanine residue comprising one or more para-hydrogen atoms is of Formula Ia.

In some embodiments, the carbon atom C¹ comprises an isotope ¹³C, wherein the isotope ¹³C is present in an amount according to its natural isotopic abundance or in an amount greater than its natural isotopic abundance. In some embodiments, Hₐ, H_{b}, or H_{c} or any other hydrogen atoms may independently be a deuterium atom, with the proviso that H_{p1*} is not a deuterium atom or a tritium atom, and H_{p2*} is not a deuterium atom or a tritium atom. In some embodiments, the para-hydrogenated peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the para-hydrogenated peptide has not been exposed to or subjected to a polarization transfer sequence.

In some embodiments, the para-hydrogenated peptide has not been exposed to or subjected to a decoupling sequence. In some embodiments, the para-hydrogenated peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In various embodiments, the natural amino acids may be represented by their name, three letter code, or one letter code as known in the art. In some embodiments, the natural amino acid is selected from alanine (Ala) (A), arginine (Arg) (R), asparagine (Asn) (N), aspartic acid (Asp) (D), cysteine (Cys) (C), glutamic acid (Glu) (E), glutamine (Gln) (Q), glycine (Gly) (G), histidine (His) (H), isoleucine (Ile) (I), leucine (Leu) (L), lysine (Lys) (K), methionine (Met) (M), phenylalanine (Phe) (F), proline (Pro) (P), serine (Ser) (S), threonine (Thr) (T), tryptophan (Trp) (W), tyrosine (Tyr) (Y), and valine (Val) (V).

In various embodiments, the amino acid is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the other amino acid(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the chiral amino acid is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from an Ala, Ile, Leu, Met, Val, Phe, Trp, Tyr, Asn, Cys, Gin, Ser, Thr, Asp, Glu, Arg, His, Lys, and Pro amino acid residue.

In various embodiments, the non-chiral amino acid is Gly. In various embodiments, the non-chiral amino acid residue is Gly. In various embodiments, the non-chiral amino acid residue is a Gly amino acid residue.

In some embodiments, one or more of the amino acids (or amino acid residues) are D-amino acids (or D-amino acid residues). In various embodiments, one or more D-amino acids are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gln, Ser, Thr, Asp, Glu, Arg, His, Lys, and Pro amino acid residues.

In various embodiments, the other amino acid residues are D-amino acids. In various embodiments, the other amino acid residues are D-amino acid residues.

In some embodiments, one or more of the amino acids (or amino acid residues) are L-amino acids (or L-amino acid residues). In some embodiments, one or more L-amino acids are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro amino acid residues.

In various embodiments, the other amino acid residues are L-amino acids. In various embodiments, the other amino acid residues are L-amino acid residues.

In various embodiments, the present invention provides a method for preparing a hyperpolarized peptide, comprising: a. providing a reagent peptide comprising at least one dehydroalanine residue and one or more other amino acid residues; b. contacting the reagent peptide with molecular hydrogen enriched with para-hydrogen and a hydrogenation catalyst under conditions effective to form a para-hydrogenated peptide; c. subjecting the para-hydrogenated peptide to a polarization transfer sequence to obtain the hyperpolarized peptide. In some embodiments, the method further comprises subjecting the para-hydrogenated peptide to a decoupling sequence.

In various embodiments, the present invention provides a method for preparing a hyperpolarized peptide, comprising: providing a reagent peptide comprising at least one dehydroalanine residue and one or more other amino acid residues; contacting the reagent peptide with molecular hydrogen enriched with para-hydrogen in the presence of a hydrogenation catalyst under conditions effective to form a para-hydrogenated peptide; subjecting the para-hydrogenated peptide to a polarization transfer sequence to obtain the hyperpolarized peptide. In some embodiments, the method further comprises subjecting the para-hydrogenated peptide to a decoupling sequence.

In various embodiments, the present invention provides a method of preparing a hyperpolarized peptide, comprising: (a) providing a reagent peptide comprising at least one dehydroalanine residue and one or more other amino acid residues; (b) contacting the reagent peptide with molecular hydrogen enriched in para-hydrogen and a hydrogenation catalyst under conditions effective to hydrogenate the dehydroalanine residue to form a para-hydrogenated peptide; and (c) subjecting the para-hydrogenated peptide to a polarization transfer sequence to obtain the hyperpolarized peptide. In some embodiments, the method further comprises subjecting the para-hydrogenated peptide to a decoupling sequence.

In various embodiments, the present invention provides a method of preparing a hyperpolarized peptide, comprising: providing a reagent peptide comprising at least one dehydroalanine residue and one or more other amino acid residues; contacting the reagent peptide with molecular hydrogen enriched in para-hydrogen in the presence of a hydrogenation catalyst under conditions effective to hydrogenate the dehydroalanine residue to form a para-hydrogenated peptide; and subjecting the para-hydrogenated peptide to a polarization transfer sequence to obtain the hyperpolarized peptide. In some embodiments, the method further comprises subjecting the para-hydrogenated peptide to a decoupling sequence.

In various embodiments, the present invention provides a method of preparing a hyperpolarized peptide, comprising: (a) providing a reagent peptide comprising at least one dehydroalanine residue and one or more other amino acid residues; (b) contacting the reagent peptide with molecular hydrogen enriched in para-hydrogen and a hydrogenation catalyst under conditions effective to hydrogenate the dehydroalanine residue to form a para-hydrogenated peptide, wherein the para-hydrogenated peptide comprises an alanine residue containing para-hydrogens; and (c) subjecting the para-hydrogenated peptide to a polarization transfer sequence.

In various embodiments, the present invention provides a method of preparing a hyperpolarized peptide, comprising: providing a reagent peptide comprising at least one dehydroalanine residue and one or more other amino acid residues; contacting the reagent peptide with molecular hydrogen enriched in para-hydrogen in the presence of a hydrogenation catalyst under conditions effective to hydrogenate the dehydroalanine residue to form a para-hydrogenated peptide, wherein the para-hydrogenated peptide comprises an alanine residue containing para-hydrogens; and subjecting the para-hydrogenated peptide to a polarization transfer sequence.

Non-limiting examples of a peptide of Formula IV, comprising at least one dehydroalanine residue and one or more other amino acid residues include:

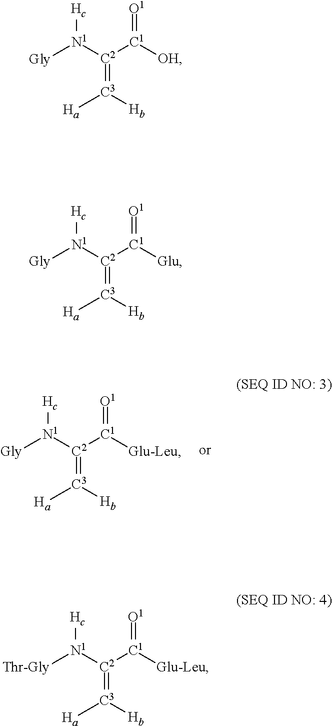

wherein C¹, C², C³, N¹, O¹, Hₐ, H_{b}, and H_{c} are as defined for Formula III or Formula IV. In some embodiments, a non-limiting example of a peptide of Formula IV is Gly-Xaa2-Glu-Leu, wherein Xaa2 is equal to Formula III. In some embodiments, a non-limiting example of a peptide of Formula IV is Thr-Gly-Xaa2-Glu-Leu, wherein Xaa2 is equal to Formula III.

A non-limiting example of a peptide of Formula IV, comprising at least one dehydroalanine residue and one or more other amino acid residues is:

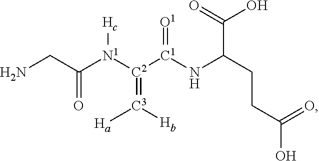

wherein C¹, C², C³, N¹, O¹, Hₐ, H_{b}, and H_{c} are as defined for Formula III or Formula IV.

In various embodiments, one or more of the hydrogen atoms in the natural amino acids or unnatural amino acids may be deuterium atoms.

In various embodiments, the present invention provides a method for preparing a para-hydrogenated peptide, comprising: providing a reagent peptide comprising at least one dehydroalanine residue and one or more other amino acid residues; and contacting the reagent peptide with molecular hydrogen enriched with para-hydrogen and a hydrogenation catalyst under conditions effective to form a para-hydrogenated peptide.

In various embodiments, the present invention provides a method for preparing a para-hydrogenated peptide, comprising: providing a reagent peptide comprising at least one dehydroalanine residue and one or more other amino acid residues; and contacting the reagent peptide with molecular hydrogen enriched with para-hydrogen in the presence of a hydrogenation catalyst under conditions effective to form a para-hydrogenated peptide.

In various embodiments, the present invention provides a reagent peptide, comprising: at least one dehydroalanine residue; and at least one other amino acid residue. In some embodiments, the at least one other amino acid residue is independently selected from any amino acid residue. In some embodiments, the at least one other amino acid residue is not a dehydroalanine residue. In some embodiments, the at least one other amino acid residue is independently selected from any amino acid. In some embodiments, the amino acids are amphoteric compounds and may exist in different ionic forms including without limitation a neutral form, a zwitterion form, a salt form, a protected form, or a combination thereof.

In various embodiments, the present invention provides a reagent peptide, comprising: at least one dehydroalanine residue; and at least one amino acid residue. In some embodiments, the at least one amino acid residue is independently selected from any amino acid residue. In some embodiments, the at least one amino acid residue is not a dehydroalanine residue. In some embodiments, the at least one amino acid residue is independently selected from any amino acid. In some embodiments, the amino acids are amphoteric compounds and may exist in different ionic forms including without limitation a neutral form, a zwitterion form, a salt form, a protected form, or a combination thereof.

Formula I-1

In various embodiments, the present invention provides a hyperpolarized peptide, comprising: at least one hyperpolarized alanine residue and one or more other amino acid residues, wherein the hyperpolarized alanine residue is of Formula I-1:

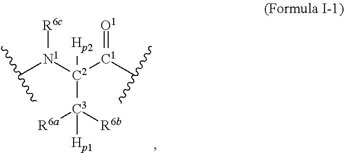

wherein, Hₚ₁ and Hₚ₂ are hydrogen atoms derived from molecular hydrogen enriched in para-hydrogen, or Hₚ₁ and Hₚ₂ are independently an ortho-hydrogen atom or a para-hydrogen atom; C¹ is a carbon atom; C² is a carbon atom; C³ is a carbon atom; N¹ is a nitrogen atom; O¹ is an oxygen atom; and R^{6a}, R^{6b} and R^{6c} are independently selected from hydrogen, deuterium, tritium and an optionally substituted substituent. In some embodiments, the one or more other amino acid residues is any natural amino acid or unnatural amino acid. In some embodiments, the one or more other amino acid residues is any natural amino acid. In some embodiments, the carbon atom C¹ comprises an isotope ¹³C, wherein the isotope ¹³C is present in an amount according to its natural isotopic abundance or in an amount greater than its natural isotopic abundance. In some embodiments, R^{6a}, R^{6b} and R^{6c} may independently be a deuterium atom, or any other hydrogen atoms may independently be a deuterium atom, with the proviso that Hₚ₁ is not a deuterium atom or a tritium atom, and Hₚ₂ is not a deuterium atom or a tritium atom. In some embodiments, the hyperpolarized peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the hyperpolarized peptide has been exposed to or subjected to a polarization transfer sequence.

In some embodiments, the hyperpolarized peptide has not been exposed to or subjected to a decoupling sequence. In some embodiments, the hyperpolarized peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, R^{6a}, R^{6b} and R^{6c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In some embodiments, the hyperpolarized peptide of Formula I-1 is any enantiomer, diastereomer, racemic mixture, enantiomerically enriched mixture, or enantiomerically pure form.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

Formula II-1

In various embodiments, the present invention provides a hyperpolarized peptide, of Formula II-1:

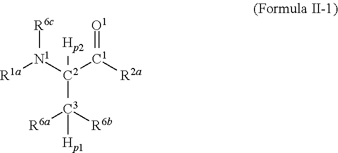

wherein, Hₚ₁ and Hₚ₂ are hydrogen atoms derived from molecular hydrogen enriched in para-hydrogen, or Hₚ₁ and Hₚ₂ are independently an ortho-hydrogen atom or a para-hydrogen atom; C¹ is a carbon atom; C² is a carbon atom; C³ is a carbon atom; N¹ is a nitrogen atom; O¹ is an oxygen atom; R^{6a}, R^{6b} and R^{6c} are independently selected from hydrogen, tritium, deuterium, and an optionally substituted substituent; R^{1a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, —C(O)—R^{3a}, and one or more other amino acid residues, wherein the one or more other amino acid residues is any natural amino acid or any unnatural amino acid, wherein R^{3a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; R^{2a} is selected from —OR^{4a} and one or more other amino acid residues, wherein the one or more other amino acid residues is any natural amino acid or any unnatural amino acid, wherein R^{4a} is absent or selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; with the proviso that one of R^{1a} or R^{2a} is one or more other amino acid residues.

In some embodiments, R^{6a}, R^{6b} and R^{6c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In some embodiments, R^{2a} is selected from N(R^{5a})(R^{5b}), wherein R^{5a} and R^{5b} are independently selected from hydrogen, deuterium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, R^{5a} and R^{5b} are optionally linked. In some embodiments, R^{2a} is selected from N(R^{5a})(R^{5b}), and R^{1a} is one or more other amino acid residues. In some embodiments, N(R^{5a})(R^{5b}) is not an amino acid residue. In some embodiments, N(R^{5a})(R^{5b}) is an amino acid residue. In some embodiments, the amino acid residue is from any natural amino acid or any unnatural amino acid. In some embodiments, the amino acid residue is from any amino acid.

In some embodiments, R^{1a} is one or more amino acid residues. In some embodiments, the one or more amino acid residues are independently selected from any amino acid. In some embodiments, the one or more amino acid residues are independently selected from any amino acid residue.

In some embodiments, R^{1a} is one or more other amino acid residues. In some embodiments, the one or more other amino acid residues are independently selected from any amino acid. In some embodiments, the one or more other amino acid residues are independently selected from any amino acid residue.

In some embodiments, R^{2a} is one or more amino acid residues. In some embodiments, the one or more amino acid residues are independently selected from any amino acid. In some embodiments, the one or more amino acid residues are independently selected from any amino acid residue.

In some embodiments, R^{2a} is one or more other amino acid residues. In some embodiments, the one or more other amino acid residues are independently selected from any amino acid. In some embodiments, the one or more other amino acid residues are independently selected from any amino acid residue.

In some embodiments, the one or more other amino acid residues is at least one other amino acid. In some embodiments, the one or more other amino acid residues is at least one other amino acid residue.

In some embodiments, the hyperpolarized peptide of Formula II-1 is any enantiomer, diastereomer, racemic mixture, enantiomerically enriched mixture, or enantiomerically pure form.

In some embodiments, the carbon atom C¹ comprises an isotope ¹³C, wherein the isotope ¹³C is present in an amount according to its natural isotopic abundance or in an amount greater than its natural isotopic abundance. In some embodiments, R^{6a}, R^{6b} and R^{6c} may independently be a deuterium atom, or any other hydrogen atoms may independently be a deuterium atom, with the proviso that Hₚ₁ is not a deuterium atom or a tritium atom, and Hₚ₂ is not a deuterium atom or a tritium atom. In some embodiments, the hyperpolarized peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the hyperpolarized peptide has been exposed to or subjected to a polarization transfer sequence.

In some embodiments, the hyperpolarized peptide has not been exposed to or subjected to a decoupling sequence. In some embodiments, the hyperpolarized peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In various embodiments, the natural amino acids may be represented by their name, three letter code, or one letter code as known in the art. In some embodiments, the natural amino acid is selected from alanine (Ala) (A), arginine (Arg) (R), asparagine (Asn) (N), aspartic acid (Asp) (D), cysteine (Cys) (C), glutamic acid (Glu) (E), glutamine (Gln) (Q), glycine (Gly) (G), histidine (His) (H), isoleucine (Ile) (I), leucine (Leu) (L), lysine (Lys) (K), methionine (Met) (M), phenylalanine (Phe) (F), proline (Pro) (P), serine (Ser) (S), threonine (Thr) (T), tryptophan (Trp) (W), tyrosine (Tyr) (Y), and valine (Val) (V).

In various embodiments, the amino acid is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the other amino acid(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the chiral amino acid is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from an Ala, Ile, Leu, Met, Val, Phe, Trp, Tyr, Asn, Cys, Gin, Ser, Thr, Asp, Glu, Arg, His, Lys, and Pro amino acid residue.

In various embodiments, the non-chiral amino acid is Gly. In various embodiments, the non-chiral amino acid residue is Gly. In various embodiments, the non-chiral amino acid residue is a Gly amino acid residue.

In some embodiments, one or more of the amino acids (or amino acid residues) are D-amino acids (or D-amino acid residues). In various embodiments, one or more D-amino acids are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro amino acid residues.

In various embodiments, the other amino acid residues are D-amino acids. In various embodiments, the other amino acid residues are D-amino acid residues.

In some embodiments, one or more of the amino acids (or amino acid residues) are L-amino acids (or L-amino acid residues). In some embodiments, one or more L-amino acids are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro amino acid residues.

In various embodiments, the other amino acid residues are L-amino acids. In various embodiments, the other amino acid residues are L-amino acid residues.

Formula III-1

In some embodiments, the dehydroalanine residue is of Formula III-1:

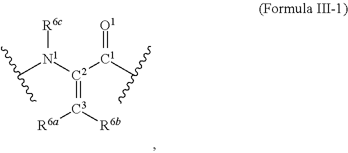

wherein, C¹ is a carbon atom; C² is a carbon atom; C³ is a carbon atom; N¹ is a nitrogen atom; O¹ is an oxygen atom; and R^{6a}, R^{6b} and R^{6c} are independently selected from hydrogen, tritium, deuterium, and an optionally substituted substituent. In some embodiments, the carbon atom C¹ comprises an isotope ¹³C, wherein the isotope ¹³C is present in an amount according to its natural isotopic abundance or in an amount greater than its natural isotopic abundance. In some embodiments, the non-hydrogen non-zero nuclear spin nucleus is ¹⁵N. In some embodiments, the ¹⁵N is present at an amount according to its natural isotopic abundance. In some embodiments, the ¹⁵N is present at a level greater than its natural isotopic abundance. In some embodiments, the amino nitrogen (or amido nitrogen or amide nitrogen) of the alanine residue comprises an isotope ¹⁵N, wherein the isotope ¹⁵N is present in an amount according to its natural isotopic abundance or in an amount greater than its natural isotopic abundance.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

In some embodiments, R^{6a}, R^{6b} and R^{6c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

Formula IV-1

In some embodiments, the peptide comprising at least one dehydroalanine residue and one or more other amino acid residues is of Formula IV-1,

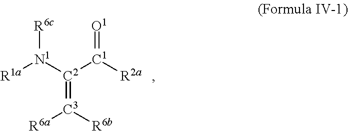

wherein, C¹ is a carbon atom; C² is a carbon atom; C³ is a carbon atom; N¹ is a nitrogen atom; O¹ is an oxygen atom; R^{6a}, R^{6b} and R^{6c} are independently selected from hydrogen, tritium, deuterium, and an optionally substituted substituent; R^{1a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, —C(O)—R^{3a}, and one or more other amino acid residues, wherein the one or more other amino acid residues is any natural amino acid or any unnatural amino acid, wherein R^{3a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; R^{2a} is selected from —OR^{4a} and one or more other amino acid residues, wherein the one or more other amino acid residues is any natural amino acid or any unnatural amino acid, wherein R^{4a} is absent or selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; with the proviso that one of R^{1a} or R^{2a} is one or more other amino acid residues. In some embodiments the peptide of Formula IV is a reagent peptide.

In some embodiments, R^{1a} is one or more amino acid residues. In some embodiments, the one or more amino acid residues are independently selected from any amino acid. In some embodiments, the one or more amino acid residues are independently selected from any amino acid residue.

In some embodiments, R^{1a} is one or more other amino acid residues. In some embodiments, the one or more other amino acid residues are independently selected from any amino acid. In some embodiments, the one or more other amino acid residues are independently selected from any amino acid residue.

In some embodiments, R^{2a} is one or more amino acid residues. In some embodiments, the one or more amino acid residues are independently selected from any amino acid. In some embodiments, the one or more amino acid residues are independently selected from any amino acid residue.

In some embodiments, R^{2a} is one or more other amino acid residues. In some embodiments, the one or more other amino acid residues are independently selected from any amino acid. In some embodiments, the one or more other amino acid residues are independently selected from any amino acid residue.

In some embodiments, the one or more other amino acid residue(s) is not a dehydroalanine residue.

In some embodiments, R^{2a} is selected from N(R^{5a})(R^{5b}), wherein R^{5a} and R^{5b} are independently selected from hydrogen, deuterium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, R^{5a} and R^{5b} are optionally linked. In some embodiments, R^{2a} is selected from N(R^{5a})(R^{5b}), and R^{1a} is one or more other amino acid residues. In some embodiments, N(R^{5a})(R^{5b}) is not an amino acid residue. In some embodiments, N(R^{5a})(R^{5b}) is an amino acid residue. In some embodiments, the amino acid residue is from any natural amino acid or any unnatural amino acid. In some embodiments, the amino acid residue is from any amino acid.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

In various embodiments, the present invention provides a para-hydrogenated peptide, comprising: at least one alanine residue comprising one or more para-hydrogen atoms and one or more other amino acid residues. In some embodiments, the one or more other amino acid residues is any natural amino acid or unnatural amino acid. In some embodiments, the one or more other amino acid residues is any natural amino acid. In some embodiments, the at least one alanine residue comprising one or more para-hydrogen atoms comprises a non-hydrogen non-zero nuclear spin nucleus. In some embodiments, the non-hydrogen non-zero nuclear spin nucleus is ¹³C. In some embodiments, the ¹³C is present at an amount according to its natural isotopic abundance. In some embodiments, the ¹³C is present at a level greater than its natural isotopic abundance. In some embodiments, the carbonyl carbon of the alanine residue comprises an isotope ¹³C, wherein the isotope ¹³C is present in an amount according to its natural isotopic abundance or in an amount greater than its natural isotopic abundance. In some embodiments, the para-hydrogenated peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In various embodiments, one or more of the hydrogen atoms of the para-hydrogenated peptide is replaced with a deuterium atom.

In some embodiments, the non-hydrogen non-zero nuclear spin nucleus is ¹⁵N. In some embodiments, the ¹⁵N is present at an amount according to its natural isotopic abundance. In some embodiments, the ¹⁵N is present at a level greater than its natural isotopic abundance. In some embodiments, the amino nitrogen (or amido nitrogen or amide nitrogen) of the alanine residue comprises an isotope ¹⁵N, wherein the isotope ¹⁵N is present in an amount according to its natural isotopic abundance or in an amount greater than its natural isotopic abundance.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, the para-hydrogenated peptide is a hyperpolarizable peptide. In some embodiments, the one or more other amino acid residue(s) is not an alanine residue comprising one or more para-hydrogen atoms.

In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

In some embodiments, R^{6a}, R^{6b} and R^{6c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

Formula Ia-1

In various embodiments, the present invention provides a para-hydrogenated peptide, comprising: at least one alanine residue comprising one or more para-hydrogen atoms and one or more other amino acid residues, wherein the alanine residue comprising one or more para-hydrogen atoms is of Formula Ia-1:

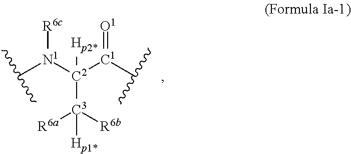

wherein, H_{p1*} and H_{p2*} are hydrogen atoms derived from molecular hydrogen enriched in para-hydrogen, or H_{p1*} and H_{p2*} are independently an ortho-hydrogen atom or a para-hydrogen atom; C¹ is a carbon atom; C² is a carbon atom; C³ is a carbon atom; N¹ is a nitrogen atom; O¹ is an oxygen atom; and R^{6a}, R^{6b} and R^{6c} are independently selected from hydrogen, deuterium, tritium and an optionally substituted substituent. In some embodiments, the one or more other amino acid residues is any natural amino acid or unnatural amino acid. In some embodiments, the one or more other amino acid residues is any natural amino acid. In some embodiments, the carbon atom C¹ comprises an isotope ¹³C, wherein the isotope ¹³C is present in an amount according to its natural isotopic abundance or in an amount greater than its natural isotopic abundance. In some embodiments, R^{6a}, R^{6b} and R^{6c} may independently be a deuterium atom, or any other hydrogen atoms may independently be a deuterium atom, with the proviso that H_{p1*} is not a deuterium atom or a tritium atom, and H_{p2*} is not a deuterium atom or a tritium atom. In some embodiments, the para-hydrogenated peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the para-hydrogenated peptide has not been exposed to or subjected to a polarization transfer sequence.

In some embodiments, the para-hydrogenated peptide has not been exposed to or subjected to a decoupling sequence. In some embodiments, the para-hydrogenated peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In some embodiments, the para-hydrogenated peptide is a hyperpolarizable peptide. In some embodiments, the one or more other amino acid residue(s) is not an alanine residue comprising one or more para-hydrogen atoms.

In some embodiments, the para-hydrogenated peptide is any enantiomer, diastereomer, racemic mixture, enantiomerically enriched mixture, or enantiomerically pure form. In some embodiments, the para-hydrogenated peptide of Formula Ia-1 is any enantiomer, diastereomer, racemic mixture, enantiomerically enriched mixture, or enantiomerically pure form.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

In some embodiments, R^{6a}, R^{6b} and R^{6c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

Formula IIa-1

In various embodiments, the present invention provides a para-hydrogenated peptide of Formula IIa-1:

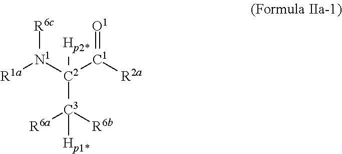

wherein, H_{p1*} and H_{p2*} are hydrogen atoms derived from molecular hydrogen enriched in para-hydrogen, or H_{p1*} and H_{p2*} are independently an ortho-hydrogen atom or a para-hydrogen atom; C¹ is a carbon atom; C² is a carbon atom; C³ is a carbon atom; N¹ is a nitrogen atom; O¹ is an oxygen atom; R^{6a}, R^{6b} and R^{6c} are independently selected from hydrogen, tritium, deuterium, and an optionally substituted substituent; R^{1a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, —C(O)—R^{3a}, and one or more other amino acid residues, wherein the one or more other amino acid residues is any natural amino acid or any unnatural amino acid, wherein R^{3a} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; R^{2a} is selected from —OR^{4a} and one or more other amino acid residues, wherein the one or more other amino acid residues is any natural amino acid or any unnatural amino acid, wherein R^{4a} is absent or selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; with the proviso that one of R^{1a} or R^{2a} is one or more other amino acid residues.

In some embodiments, R^{6a}, R^{6b} and R^{6c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In some embodiments, R^{2a} is selected from N(R^{5a})(R^{5b}), wherein R^{5a} and R^{5b} are independently selected from hydrogen, deuterium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, R^{5a} and R^{5b} are optionally linked. In some embodiments, R^{2a} is selected from N(R^{5a})(R^{5b}), and R^{1a} is one or more other amino acid residues. In some embodiments, N(R^{5a})(R^{5b}) is not an amino acid residue. In some embodiments, N(R^{5a})(R^{5b}) is an amino acid residue. In some embodiments, the amino acid residue is from any natural amino acid or any unnatural amino acid. In some embodiments, the amino acid residue is from any amino acid.

In some embodiments, R^{1a} is one or more amino acid residues. In some embodiments, the one or more amino acid residues are independently selected from any amino acid. In some embodiments, the one or more amino acid residues are independently selected from any amino acid residue.

In some embodiments, R^{1a} is one or more other amino acid residues. In some embodiments, the one or more other amino acid residues are independently selected from any amino acid. In some embodiments, the one or more other amino acid residues are independently selected from any amino acid residue.

In some embodiments, R^{2a} is one or more amino acid residues. In some embodiments, the one or more amino acid residues are independently selected from any amino acid. In some embodiments, the one or more amino acid residues are independently selected from any amino acid residue.

In some embodiments, R^{2a} is one or more other amino acid residues. In some embodiments, the one or more other amino acid residues are independently selected from any amino acid. In some embodiments, the one or more other amino acid residues are independently selected from any amino acid residue.

In some embodiments, the para-hydrogenated peptide of Formula IIa-1 is any enantiomer, diastereomer, racemic mixture, enantiomerically enriched mixture, or enantiomerically pure form.

In some embodiments, the para-hydrogenated peptide is a hyperpolarizable peptide. In some embodiments, the one or more other amino acid residue(s) is not an alanine residue comprising one or more para-hydrogen atoms. In some embodiments, the one or more other amino acid residue(s) is optionally an alanine residue comprising one or more para-hydrogen atoms. In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms. In some embodiments, the alanine residue comprising one or more para-hydrogen atoms is of Formula Ia-1.

In some embodiments, the carbon atom C¹ comprises an isotope ¹³C, wherein the isotope ¹³C is present in an amount according to its natural isotopic abundance or in an amount greater than its natural isotopic abundance. In some embodiments, R^{6a}, R^{6b} and R^{6c} may independently be a deuterium atom, or any other hydrogen atoms may independently be a deuterium atom, with the proviso that H_{p1*} is not a deuterium atom or a tritium atom, and H_{p2*} is not a deuterium atom or a tritium atom. In some embodiments, the para-hydrogenated peptide is an amphoteric compound and may exist in different ionic forms including without limitation a neutral form, a zwitterion form, a salt form, a protected form, or a combination thereof. In some embodiments, the natural amino acids are amphoteric compounds and may exist in different ionic forms including without limitation a neutral form, a zwitterion form, a salt form, a protected form, or a combination thereof. In some embodiments, the unnatural amino acids are amphoteric compounds and may exist in different ionic forms including without limitation a neutral form, a zwitterion form, a salt form, a protected form, or a combination thereof. In some embodiments, one or more hydrogen atoms in the natural amino acids or unnatural amino acids may be deuterium atoms. In some embodiments, the para-hydrogenated peptide has not been exposed to or subjected to a polarization transfer sequence.

In some embodiments, the para-hydrogenated peptide has not been exposed to or subjected to a decoupling sequence. In some embodiments, the para-hydrogenated peptide has been exposed to or subjected to a decoupling sequence.

In some embodiments, the nitrogen atom N¹ comprises an isotope ¹⁵N, wherein the isotope ¹⁵N is present in an amount according to its natural isotopic abundance or in an amount greater than its natural isotopic abundance.

In various embodiments, the natural amino acids may be represented by their name, three letter code, or one letter code as known in the art. In some embodiments, the natural amino acid is selected from alanine (Ala) (A), arginine (Arg) (R), asparagine (Asn) (N), aspartic acid (Asp) (D), cysteine (Cys) (C), glutamic acid (Glu) (E), glutamine (Gln) (Q), glycine (Gly) (G), histidine (His) (H), isoleucine (Ile) (I), leucine (Leu) (L), lysine (Lys) (K), methionine (Met) (M), phenylalanine (Phe) (F), proline (Pro) (P), serine (Ser) (S), threonine (Thr) (T), tryptophan (Trp) (W), tyrosine (Tyr) (Y), and valine (Val) (V).

In various embodiments, the amino acid is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly. In various embodiments, the amino acid residue is selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the other amino acid(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly. In various embodiments, the other amino acid residue(s) is independently selected from Ala, Ile, Leu, Met, Val, Phe, Trp, Tyr, Asn, Cys, Gin, Ser, Thr, Asp, Glu, Arg, His, Lys, Pro, and Gly amino acid residue.

In various embodiments, the chiral amino acid is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from Ala, Ile, Leu, Met, Val, Phe, Trp, Tyr, Asn, Cys, Gin, Ser, Thr, Asp, Glu, Arg, His, Lys, and Pro. In various embodiments, the chiral amino acid residue is selected from an Ala, Ile, Leu, Met, Val, Phe, Trp, Tyr, Asn, Cys, Gin, Ser, Thr, Asp, Glu, Arg, His, Lys, and Pro amino acid residue.

In various embodiments, the non-chiral amino acid is Gly. In various embodiments, the non-chiral amino acid residue is Gly. In various embodiments, the non-chiral amino acid residue is a Gly amino acid residue.

In some embodiments, one or more of the amino acids (or amino acid residues) are D-amino acids (or D-amino acid residues). In various embodiments, one or more D-amino acids are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro. In various embodiments, one or more D-amino acid residues are selected from the group consisting of D-enantiomers of Ala, Ile, Leu, Met, Val, Phe, Trp, Tyr, Asn, Cys, Gin, Ser, Thr, Asp, Glu, Arg, His, Lys, and Pro amino acid residues.

In various embodiments, the other amino acid residues are D-amino acids. In various embodiments, the other amino acid residues are D-amino acid residues.

In some embodiments, one or more of the amino acids (or amino acid residues) are L-amino acids (or L-amino acid residues). In some embodiments, one or more L-amino acids are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro. In some embodiments, one or more L-amino acid residues are selected from the group consisting of L-enantiomers of Ala, Ile, Leu, Met, Val, Phe, Trp, Tyr, Asn, Cys, Gin, Ser, Thr, Asp, Glu, Arg, His, Lys, and Pro amino acid residues.

In various embodiments, the other amino acid residues are L-amino acids. In various embodiments, the other amino acid residues are L-amino acid residues.

In some embodiments, the one or more other amino acid residues are independently selected from any amino acid. In some embodiments, the one or more other amino acid residues are independently selected from any amino acid residue. In some embodiments, the one or more other amino acid residues is at least one other amino acid. In some embodiments, the one or more other amino acid residues is at least one other amino acid residue.

Hyperpolarized Compounds

In various embodiments, the present invention provides a hyperpolarized compound, comprising a hyperpolarized alanine residue.

In various embodiment, the present invention provides a hyperpolarized compound, comprising at least one hyperpolarized alanine residue.

In some embodiments, the hyperpolarized compound is any enantiomer, diastereomer, racemic mixture, enantiomerically enriched mixture, or enantiomerically pure form.

In various embodiments, the present invention provides a hyperpolarized compound, comprising a hyperpolarized alanine residue of Formula V:

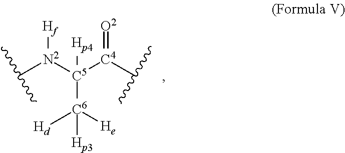

wherein, Hₚ₃ and Hₚ₄ are hydrogen atoms derived from molecular hydrogen enriched in para-hydrogen, or Hₚ₃ and Hₚ₄ are independently an ortho-hydrogen atom or a para-hydrogen atom; C⁴ is a carbon atom; C⁵ is a carbon atom; C⁶ is a carbon atom; N² is a nitrogen atom; O² is an oxygen atom; H_{d} is a hydrogen atom or a deuterium atom; Hₑ is a hydrogen atom or a deuterium atom; and H_{f} is a hydrogen atom or a deuterium atom. In some embodiments, the carbon atom C⁴ comprises an isotope ¹³C, wherein the isotope ¹³C is present in an amount according to its natural isotopic abundance or in an amount greater than its natural isotopic abundance. In some embodiments, H_{d}, Hₑ, or H_{f} or any other hydrogen atoms may independently be deuterium atoms, with the proviso that Hₚ₃ is not a deuterium atom or a tritium atom, and Hₚ₄ is not a deuterium atom or a tritium atom.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, a hyperpolarized compound comprises a hyperpolarized alanine residue of Formula V, wherein the hyperpolarized compound is any enantiomer, diastereomer, racemic mixture, enantiomerically enriched mixture, or enantiomerically pure form.

In various embodiments, the present invention provides a hyperpolarized compound, of Formula VI:

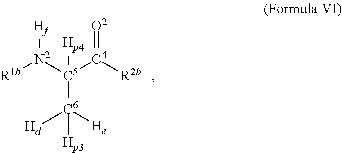

wherein, Hₚ₃ and Hₚ₄ are hydrogen atoms derived from molecular hydrogen enriched in para-hydrogen, or Hₚ₃ and Hₚ₄ are independently an ortho-hydrogen atom or a para-hydrogen atom; C⁴ is a carbon atom; C⁵ is a carbon atom; C⁶ is a carbon atom; N² is a nitrogen atom; O² is an oxygen atom; H_{d} is a hydrogen atom or a deuterium atom; Hₑ is a hydrogen atom or a deuterium atom; H_{f} is a hydrogen atom or a deuterium atom; R^{1b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —C(O)—R^{3b}; R^{2b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —O—R^{4b}; R^{3b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; and R^{4b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, the carbon atom C⁴ comprises an isotope ¹³C, wherein the isotope ¹³C is present in an amount according to its natural isotopic abundance or in an amount greater than its natural isotopic abundance. In some embodiments, H_{d}, Hₑ, or H_{f} or any other hydrogen atoms may independently be deuterium atoms, with the proviso that Hₚ₃ is not a deuterium atom or a tritium atom, and Hₚ₄ is not a deuterium atom or a tritium atom. In some embodiments, the hyperpolarized compound of Formula VI is:

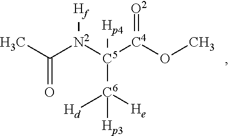

wherein Hₚ₃, Hₚ₄, C⁴, C⁵, C⁶, N², O², H_{d}, Hₑ, and H_{f} are as defined for Formula VI.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, the hyperpolarized compound of Formula VI is any enantiomer, diastereomer, racemic mixture, enantiomerically enriched mixture, or enantiomerically pure form.

In various embodiments, the present invention provides a method of preparing a hyperpolarized compound, comprising: (a) providing a reagent compound comprising at least one dehydroalanine residue; (b) contacting the reagent compound with molecular hydrogen enriched in para-hydrogen and a hydrogenation catalyst under conditions effective to hydrogenate the dehydroalanine residue to form a para-hydrogenated compound, wherein the para-hydrogenated compound comprises an alanine residue comprising one or more para-hydrogen atoms; and (c) subjecting the para-hydrogenated compound to a polarization transfer sequence. In some embodiments, the method further comprises subjecting the para-hydrogenated compound to a decoupling sequence.

In various embodiments, the present invention provides a method of preparing a hyperpolarized compound, comprising: providing a reagent compound comprising at least one dehydroalanine residue; contacting the reagent compound with molecular hydrogen enriched in para-hydrogen in the presence of a hydrogenation catalyst under conditions effective to hydrogenate the dehydroalanine residue to form a para-hydrogenated compound, wherein the para-hydrogenated compound comprises an alanine residue comprising one or more para-hydrogen atoms; and subjecting the para-hydrogenated compound to a polarization transfer sequence. In some embodiments, the method further comprises subjecting the para-hydrogenated compound to a decoupling sequence.

In various embodiments, the present invention provides a method for preparing a hyperpolarized compound, comprising: a. providing a reagent compound comprising at least one dehydroalanine residue; b. contacting the reagent compound with molecular hydrogen enriched with para-hydrogen and a hydrogenation catalyst under conditions effective to form a para-hydrogenated compound; c. subjecting the para-hydrogenated compound to a polarization transfer sequence to obtain the hyperpolarized compound. In some embodiments, the method further comprises subjecting the para-hydrogenated compound to a decoupling sequence.

In various embodiments, the present invention provides a method for preparing a hyperpolarized compound, comprising: providing a reagent compound comprising at least one dehydroalanine residue; contacting the reagent compound with molecular hydrogen enriched with para-hydrogen in the presence of a hydrogenation catalyst under conditions effective to form a para-hydrogenated compound; subjecting the para-hydrogenated compound to a polarization transfer sequence to obtain the hyperpolarized compound. In some embodiments, the method further comprises subjecting the para-hydrogenated compound to a decoupling sequence.

In some embodiments, the alanine residue comprising one or more para-hydrogen atoms comprises a non-hydrogen non-zero nuclear spin nucleus. In some embodiments, the non-hydrogen non-zero nuclear spin nucleus is ¹³C. In some embodiments, the ¹³C is present at an amount according to its natural isotopic abundance. In some embodiments, the ¹³C is present at a level greater than its natural isotopic abundance. In some embodiments, the carbonyl carbon of the alanine residue comprising one or more para-hydrogen atoms comprises an isotope ¹³C, wherein the isotope ¹³C is present in an amount according to its natural isotopic abundance or in an amount greater than its natural isotopic abundance. In some embodiments, the hyperpolarized compound is an amphoteric compound and may exist in different ionic forms including without limitation a neutral form, a zwitterion form, a salt form, a protected form, or a combination thereof. In some embodiments, the hyperpolarized compound comprises a hyperpolarized alanine residue.

In some embodiments, the non-hydrogen non-zero nuclear spin nucleus is ¹⁵N. In some embodiments, the ¹⁵N is present at an amount according to its natural isotopic abundance. In some embodiments, the ¹⁵N is present at a level greater than its natural isotopic abundance. In some embodiments, the amino nitrogen (or amido nitrogen or amide nitrogen) of the alanine residue comprising one or more para-hydrogen atoms comprises an isotope ¹⁵N, wherein the isotope ¹⁵N is present in an amount according to its natural isotopic abundance or in an amount greater than its natural isotopic abundance.

In some embodiments, the hyperpolarized compound has been exposed to or subjected to a polarization transfer sequence.

In some embodiments, the hyperpolarized compound has not been exposed to or subjected to a decoupling sequence. In some embodiments, the hyperpolarized compound has been exposed to or subjected to a decoupling sequence.

In various embodiments, the present invention provides a para-hydrogenated compound of Formula VIa:

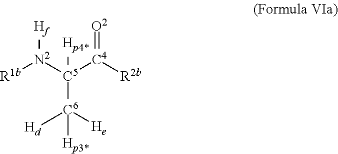

wherein, H_{p3*} and H_{p4*} are hydrogen atoms derived from molecular hydrogen enriched in para-hydrogen, or H_{p3*} and H_{p4*} are independently an ortho-hydrogen atom or a para-hydrogen atom; C⁴ is a carbon atom; C⁵ is a carbon atom; C⁶ is a carbon atom; N² is a nitrogen atom; O² is an oxygen atom; H_{d} is a hydrogen atom or a deuterium atom; Hₑ is a hydrogen atom or a deuterium atom; H_{f} is a hydrogen atom or a deuterium atom; R^{1b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —C(O)—R^{3b}; R^{2b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —O—R^{4b}; R^{3b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; and R^{4b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, the carbon atom C⁴ comprises an isotope ¹³C, wherein the isotope ¹³C is present in an amount according to its natural isotopic abundance or in an amount greater than its natural isotopic abundance. In some embodiments, H_{d}, Hₑ, or H_{f} or any other hydrogen atoms may independently be deuterium atoms, with the proviso that H_{p3*} is not a deuterium atom or a tritium atom, and H_{p4*} is not a deuterium atom or a tritium atom. In some embodiments, the para-hydrogenated compound is an amphoteric compound and may exist in different ionic forms including without limitation a neutral form, a zwitterion form, a salt form, a protected form, or a combination thereof. In some embodiments, the para-hydrogenated compound has not been exposed to or subjected to a polarization transfer sequence. In some embodiments, the para-hydrogenated compound of Formula VIa is:

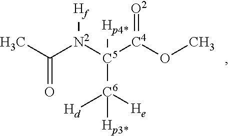

wherein H_{p3*}, H_{p4*}, C⁴, C⁵, C⁶, N², O², H_{d}, Hₑ, and H_{f} are as defined for Formula VIa.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, the para-hydrogenated compound is any enantiomer, diastereomer, racemic mixture, enantiomerically enriched mixture, or enantiomerically pure form. In some embodiments, the para-hydrogenated compound of Formula VIa is any enantiomer, diastereomer, racemic mixture, enantiomerically enriched mixture, or enantiomerically pure form.

In some embodiments, the para-hydrogenated compound is a hyperpolarizable compound. In some embodiments, the para-hydrogenated compound of Formula VIa is a hyperpolarizable compound.

In some embodiments, the dehydroalanine residue is of Formula VII:

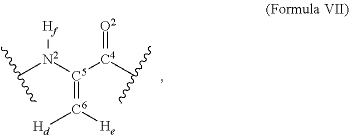

wherein, C⁴ is a carbon atom; C⁵ is a carbon atom; C⁶ is a carbon atom; N² is a nitrogen atom; O² is an oxygen atom; H_{d} is a hydrogen atom or a deuterium atom; Hₑ is a hydrogen atom or a deuterium atom; and H_{f} is a hydrogen atom or a deuterium atom. In some embodiments, the carbon atom C⁴ comprises an isotope ¹³C, wherein the isotope ¹³C is present in an amount according to its natural isotopic abundance or in an amount greater than its natural isotopic abundance. In some embodiments, H_{d}, Hₑ, or H_{f} may independently be deuterium atoms.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, the compound comprising at least one dehydroalanine residue is of Formula VIII,

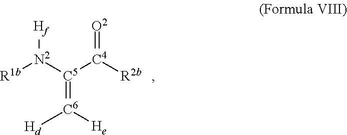

wherein, C⁴ is a carbon atom; C⁵ is a carbon atom; C⁶ is a carbon atom; N² is a nitrogen atom; O² is an oxygen atom; H_{d} is a hydrogen atom or a deuterium atom; Hₑ is a hydrogen atom or a deuterium atom; H_{f} is a hydrogen atom or a deuterium atom; R^{1b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —C(O)—R^{3b}; R^{2b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —O—R^{4b}; R^{3b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; and R^{4b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, the compound of Formula VIII is a reagent compound.

In some embodiments, the compound of Formula VIII, the carbon atom C⁴ comprises an isotope ¹³C, wherein the isotope ¹³C is present in an amount according to its natural isotopic abundance or in an amount greater than its natural isotopic abundance. In some embodiments, H_{d}, Hₑ, or H_{f} or any other hydrogen atoms may independently be deuterium atoms.

In some embodiments, the compound of Formula VIII, the nitrogen atom N² comprises an isotope ¹⁵N, wherein the isotope ¹⁵N is present in an amount according to its natural isotopic abundance or in an amount greater than its natural isotopic abundance.

In some embodiments, the compound of Formula VIII comprising at least one dehydroalanine residue is an amphoteric compound and may exist in different ionic forms including without limitation a neutral form, a zwitterion form, a salt form, a protected form, or a combination thereof.

Formula V-1

In various embodiments, the present invention provides a hyperpolarized compound, comprising a hyperpolarized alanine residue of Formula V-1:

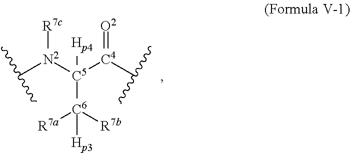

wherein, Hₚ₃ and Hₚ₄ are hydrogen atoms derived from molecular hydrogen enriched in para-hydrogen, or Hₚ₃ and Hₚ₄ are independently an ortho-hydrogen atom or a para-hydrogen atom; C⁴ is a carbon atom; C⁵ is a carbon atom; C⁶ is a carbon atom; N² is a nitrogen atom; O² is an oxygen atom; and R^{7a}, R^{7b} and R^{7c} are independently selected from hydrogen, deuterium, tritium and an optionally substituted substituent. In some embodiments, the carbon atom C⁴ comprises an isotope ¹³C, wherein the isotope ¹³C is present in an amount according to its natural isotopic abundance or in an amount greater than its natural isotopic abundance. In some embodiments, R^{7a}, R^{7b} and R^{7c} may independently be a deuterium atom, or any other hydrogen atoms may independently be deuterium atoms, with the proviso that Hₚ₃ is not a deuterium atom or a tritium atom, and Hₚ₄ is not a deuterium atom or a tritium atom.

In some embodiments, R^{7a}, R^{7b} and R^{7c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, a hyperpolarized compound comprises a hyperpolarized alanine residue of Formula V-1, wherein the hyperpolarized compound is any enantiomer, diastereomer, racemic mixture, enantiomerically enriched mixture, or enantiomerically pure form.

In some embodiments, the hyperpolarized compound has been exposed to or subjected to a polarization transfer sequence.

In some embodiments, the hyperpolarized compound has not been exposed to or subjected to a decoupling sequence. In some embodiments, the hyperpolarized compound has been exposed to or subjected to a decoupling sequence.

Formula VI-1

In various embodiments, the present invention provides a hyperpolarized compound, of Formula VI-1:

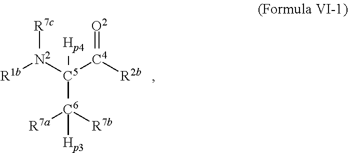

wherein, Hₚ₃ and Hₚ₄ are hydrogen atoms derived from molecular hydrogen enriched in para-hydrogen, or Hₚ₃ and Hₚ₄ are independently an ortho-hydrogen atom or a para-hydrogen atom; C⁴ is a carbon atom; C⁵ is a carbon atom; C⁶ is a carbon atom; N² is a nitrogen atom; O² is an oxygen atom; R^{7a}, R^{7b} and R^{7c} are independently selected from hydrogen, tritium, deuterium, and an optionally substituted substituent; R^{1b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —C(O)—R^{3b}; R^{2b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —O—R^{4b}; R^{3b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; and R^{4b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, the carbon atom C⁴ comprises an isotope ¹³C, wherein the isotope ¹³C is present in an amount according to its natural isotopic abundance or in an amount greater than its natural isotopic abundance. In some embodiments, R^{7a}, R^{7b} and R^{7c} may independently be a deuterium atom, or any other hydrogen atoms may independently be deuterium atoms, with the proviso that Hₚ₃ is not a deuterium atom or a tritium atom, and Hₚ₄ is not a deuterium atom or a tritium atom.

In some embodiments, R^{7a}, R^{7b} and R^{7c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, the hyperpolarized compound of Formula VI-1 is any enantiomer, diastereomer, racemic mixture, enantiomerically enriched mixture, or enantiomerically pure form.

In some embodiments, the hyperpolarized compound has been exposed to or subjected to a polarization transfer sequence.

In some embodiments, the hyperpolarized compound has not been exposed to or subjected to a decoupling sequence. In some embodiments, the hyperpolarized compound has been exposed to or subjected to a decoupling sequence.

Formula VIa-1

In various embodiments, the present invention provides a para-hydrogenated compound of Formula VIa-1:

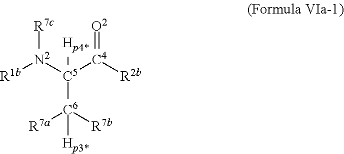

wherein, H_{p3*} and H_{p4*} are hydrogen atoms derived from molecular hydrogen enriched in para-hydrogen, or H_{p3*} and H_{p4*} are independently an ortho-hydrogen atom or a para-hydrogen atom; C⁴ is a carbon atom; C⁵ is a carbon atom; C⁶ is a carbon atom; N² is a nitrogen atom; O² is an oxygen atom; R^{7a}, R^{7b} and R^{7c} are independently selected from hydrogen, tritium, deuterium, and an optionally substituted substituent; R^{1b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —C(O)—R^{3b}; R^{2b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —O—R^{4b}; R^{3b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; and R^{4b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, the carbon atom C⁴ comprises an isotope ¹³C, wherein the isotope ¹³C is present in an amount according to its natural isotopic abundance or in an amount greater than its natural isotopic abundance. In some embodiments, R^{7a}, R^{7b} and R^{7c} may independently be a deuterium atom, or any other hydrogen atoms may independently be deuterium atoms, with the proviso that H_{p3*} is not a deuterium atom or a tritium atom, and H_{p4*} is not a deuterium atom or a tritium atom. In some embodiments, the para-hydrogenated compound is an amphoteric compound and may exist in different ionic forms including without limitation a neutral form, a zwitterion form, a salt form, a protected form, or a combination thereof. In some embodiments, the para-hydrogenated compound has not been exposed to or subjected to a polarization transfer sequence.

In some embodiments, the para-hydrogenated compound has not been exposed to or subjected to a decoupling sequence. In some embodiments, the para-hydrogenated compound has been exposed to or subjected to a decoupling sequence.

In some embodiments, R^{7a}, R^{7b} and R^{7c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, the para-hydrogenated compound is any enantiomer, diastereomer, racemic mixture, enantiomerically enriched mixture, or enantiomerically pure form. In some embodiments, the para-hydrogenated compound of Formula VIa-1 is any enantiomer, diastereomer, racemic mixture, enantiomerically enriched mixture, or enantiomerically pure form.

In some embodiments, the para-hydrogenated compound is a hyperpolarizable compound. In some embodiments, the para-hydrogenated compound of Formula VIa-1 is a hyperpolarizable compound.

Formula VII-1

In some embodiments, the dehydroalanine residue is of Formula VII-1:

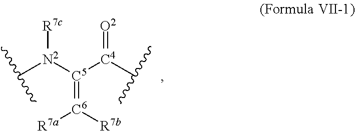

wherein, C⁴ is a carbon atom; C⁵ is a carbon atom; C⁶ is a carbon atom; N² is a nitrogen atom; O² is an oxygen atom; and R^{7a}, R^{7b} and R^{7c} are independently selected from hydrogen, deuterium, tritium and an optionally substituted substituent. In some embodiments, the carbon atom C⁴ comprises an isotope ¹³C, wherein the isotope ¹³C is present in an amount according to its natural isotopic abundance or in an amount greater than its natural isotopic abundance. In some embodiments, R^{7a}, R^{7b} and R^{7c} may independently be a deuterium atom.

In some embodiments, the nitrogen atom N² comprises an isotope ¹⁵N, wherein the isotope ¹⁵N is present in an amount according to its natural isotopic abundance or in an amount greater than its natural isotopic abundance.

In some embodiments, R^{7a}, R^{7b} and R^{7c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In some embodiments, the compound comprising at least one dehydroalanine residue is of Formula VIII-1,

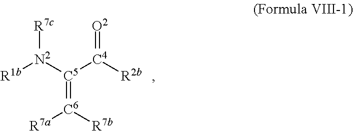

wherein, C⁴ is a carbon atom; C⁵ is a carbon atom; C⁶ is a carbon atom; N² is a nitrogen atom; O² is an oxygen atom; R^{7a}, R^{7b} and R^{7c} are independently selected from hydrogen, deuterium, tritium and an optionally substituted substituent; R^{1b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —C(O)—R^{3b}; R^{2b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, a protecting group, and —O—R^{4b}; R^{3b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group; and R^{4b} is selected from hydrogen, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group. In some embodiments, the compound of Formula VIII-1 is a reagent compound.

In some embodiments, the compound of Formula VIII-1, the carbon atom C⁴ comprises an isotope ¹³C, wherein the isotope ¹³C is present in an amount according to its natural isotopic abundance or in an amount greater than its natural isotopic abundance. In some embodiments, H_{d}, Hₑ, or H_{f} or any other hydrogen atoms may independently be deuterium atoms.

In some embodiments, the compound of Formula VIII-1, the nitrogen atom N² comprises an isotope ¹⁵N, wherein the isotope ¹⁵N is present in an amount according to its natural isotopic abundance or in an amount greater than its natural isotopic abundance.

In some embodiments, the compound of Formula VIII-1 comprising at least one dehydroalanine residue is an amphoteric compound and may exist in different ionic forms including without limitation a neutral form, a zwitterion form, a salt form, a protected form, or a combination thereof.

In some embodiments, R^{7a}, R^{7b} and R^{7c} are independently selected from hydrogen, deuterium, tritium, alkyl, substituted alkyl, aryl, substituted aryl, heteroalkyl, substituted heteroalkyl, heteroaryl, substituted heteroaryl, cyclyl, substituted cyclyl, heterocyclyl, substituted heterocyclyl, and a protecting group.

In various embodiments, the present invention provides a method for preparing a para-hydrogenated compound, comprising: providing a reagent compound comprising at least one dehydroalanine residue; and contacting the reagent compound with molecular hydrogen enriched with para-hydrogen and a hydrogenation catalyst under conditions effective to form a para-hydrogenated compound.

In various embodiments, the present invention provides a method for preparing a para-hydrogenated compound, comprising: providing a reagent compound comprising at least one dehydroalanine residue; and contacting the reagent compound with molecular hydrogen enriched with para-hydrogen in the presence of a hydrogenation catalyst under conditions effective to form a para-hydrogenated compound.

### Various Non-Limiting Embodiments

In various embodiments, one or more is at least one.

Hyperpolarized Peptide

In various embodiments, the present invention provides a hyperpolarized peptide, comprising: at least one hyperpolarized alanine residue; and at least one other amino acid residue. In some embodiments, the at least one other amino acid residue is independently selected from any amino acid residue. In some embodiments, the at least one other amino acid residue is independently selected from any natural amino acid residue and any unnatural amino acid residue. In some embodiments, the at least one other amino acid residue is independently selected from any natural amino acid residue. In some embodiments, the at least one other amino acid residue is not a hyperpolarized alanine residue. In some embodiments, the at least one other amino acid residue is independently selected from any amino acid. In some embodiments, the at least one other amino acid residue is independently selected from any natural amino acid and any unnatural amino acid. In some embodiments, the at least one other amino acid residue is independently selected from any natural amino acid. In some embodiments, the amino acids are amphoteric compounds and may exist in different ionic forms including without limitation a neutral form, a zwitterion form, a salt form, a protected form, or a combination thereof.

In various embodiments, the present invention provides a hyperpolarized peptide, comprising: at least one hyperpolarized alanine residue; and at least one amino acid residue. In some embodiments, the at least one amino acid residue is independently selected from any amino acid residue. In some embodiments, the at least one amino acid residue is independently selected from any natural amino acid residue and any unnatural amino acid residue. In some embodiments, the at least one amino acid residue is independently selected from any natural amino acid residue. In some embodiments, the at least one amino acid residue is not a hyperpolarized alanine residue. In some embodiments, the at least one amino acid residue is independently selected from any amino acid. In some embodiments, the at least one amino acid residue is independently selected from any natural amino acid and any unnatural amino acid. In some embodiments, the at least one amino acid residue is independently selected from any natural amino acid. In some embodiments, the amino acids are amphoteric compounds and may exist in different ionic forms including without limitation a neutral form, a zwitterion form, a salt form, a protected form, or a combination thereof.

Reagent Peptide

In various embodiments, the present invention provides a reagent peptide, comprising: at least one dehydroalanine residue; and at least one other amino acid residue. In some embodiments, the at least one other amino acid residue is independently selected from any amino acid residue. In some embodiments, the at least one other amino acid residue is not a dehydroalanine residue. In some embodiments, the at least one other amino acid residue is independently selected from any amino acid. In some embodiments, the amino acids are amphoteric compounds and may exist in different ionic forms including without limitation a neutral form, a zwitterion form, a salt form, a protected form, or a combination thereof.

In various embodiments, the present invention provides a reagent peptide, comprising: at least one dehydroalanine residue; and at least one amino acid residue. In some embodiments, the at least one amino acid residue is independently selected from any amino acid residue. In some embodiments, the at least one amino acid residue is not a dehydroalanine residue. In some embodiments, the at least one amino acid residue is independently selected from any amino acid. In some embodiments, the amino acids are amphoteric compounds and may exist in different ionic forms including without limitation a neutral form, a zwitterion form, a salt form, a protected form, or a combination thereof.

Para-Hydrogenated Compound

In various embodiments, the present invention provides a para-hydrogenated compound, comprising: an alanine residue comprising one or more para-hydrogen atoms. In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms.

Para-Hydrogenated Peptide

In various embodiments, the present invention provides a para-hydrogenated peptide, comprising: at least one alanine residue comprising one or more para-hydrogen atoms; and one or more other amino acid residues. In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms. In some embodiments, the one or more other amino acid residues is not an alanine residue comprising one or more para-hydrogen atoms. In some embodiments, the one or more other amino acid residues are independently selected from any amino acid residue. In some embodiments, the one or more other amino acid residues are independently selected from any amino acid. In some embodiments, the amino acids are amphoteric compounds and may exist in different ionic forms including without limitation a neutral form, a zwitterion form, a salt form, a protected form, or a combination thereof.

In various embodiments, the present invention provides a para-hydrogenated peptide, comprising: at least one alanine residue comprising one or more para-hydrogen atoms; and at least one other amino acid residue. In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms. In some embodiments, the at least one other amino acid residue is not an alanine residue comprising one or more para-hydrogen atoms. In some embodiments, the at least one other amino acid residue is independently selected from any natural amino acid residue and any unnatural amino acid residue. In some embodiments, the at least one other amino acid residue is independently selected from any natural amino acid residue. In some embodiments, the at least one other amino acid residue is independently selected from any amino acid. In some embodiments, the at least one other amino acid residue is independently selected from any natural amino acid and any unnatural amino acid. In some embodiments, the at least one other amino acid residue is independently selected from any natural amino acid. In some embodiments, the amino acids are amphoteric compounds and may exist in different ionic forms including without limitation a neutral form, a zwitterion form, a salt form, a protected form, or a combination thereof.

In various embodiments, the present invention provides a para-hydrogenated peptide, comprising: at least one alanine residue comprising one or more para-hydrogen atoms; and at least one amino acid residue. In some embodiments, the alanine residue comprising one or more para-hydrogen atoms is an alanine residue comprising one para-hydrogen atom. In some embodiments, the alanine residue comprising one or more para-hydrogen atoms is an alanine residue comprising two para-hydrogen atoms. In some embodiments, the at least one amino acid residue is not an alanine residue comprising one or more para-hydrogen atoms. In some embodiments, the at least one amino acid residue is independently selected from any amino acid residue. In some embodiments, the at least one amino acid residue is independently selected from any natural amino acid residue and any unnatural amino acid residue. In some embodiments, the at least one amino acid residue is independently selected from any natural amino acid residue. In some embodiments, the at least one amino acid residue is independently selected from any amino acid. In some embodiments, the at least one amino acid residue is independently selected from any natural amino acid and any unnatural amino acid. In some embodiments, the at least one amino acid residue is independently selected from any natural amino acid. In some embodiments, the amino acids are amphoteric compounds and may exist in different ionic forms including without limitation a neutral form, a zwitterion form, a salt form, a protected form, or a combination thereof.

Hyperpolarized Compound

In various embodiment, the present invention provides a hyperpolarized compound, comprising at least one hyperpolarized alanine residue.

Reagent Compound

In various embodiments, the present invention provides a reagent compound, comprising at least one dehydroalanine residue.

### Various Methods of the Invention

Imaging Methods

In various embodiments, the present invention provides a method for obtaining one or more magnetic resonance images of a subject, comprising: providing a regent peptide comprising at least one dehydroalanine residue and one or more other amino acid residues; contacting the reagent peptide with molecular hydrogen enriched with parahydrogen in the presence of a hydrogenation catalyst under conditions effective to form a para-hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; and generating one or more magnetic resonance images of the subject from the detected magnetic resonance signals.

In various embodiments, the present invention provides a method for obtaining one or more magnetic resonance images of a subject, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; and generating one or more magnetic resonance images of the subject from the detected magnetic resonance signals.

In various embodiments, the present invention provides a method of imaging a subject, comprising: providing a hyperpolarized peptide, wherein the hyperpolarized peptide comprises at least one hyperpolarized alanine residue and one or more other amino acid residues; administering the hyperpolarized peptide to the subject; and imaging the subject after administering the hyperpolarized peptide to the subject. In some embodiments, imaging the subject comprises magnetic resonance imaging (MRI) of the subject.

In various embodiments, the present invention provides a method for obtaining one or more magnetic resonance images of a subject, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; and generating one or more magnetic resonance images of the subject from the detected magnetic resonance signals.

In various embodiments, the present invention provides a method of imaging a subject, comprising: providing a hyperpolarized compound, wherein the hyperpolarized compound comprises at least one hyperpolarized alanine residue; administering the hyperpolarized compound to the subject; and imaging the subject after administering the hyperpolarized compound to the subject. In some embodiments, imaging the subject comprises magnetic resonance imaging (MRI) of the subject.

Diagnostic Methods

In various embodiments, the present invention provides a method for diagnosing a disease in a subject, comprising: providing a reagent peptide comprising at least one dehydroalanine residue and one or more other amino acid residues; contacting the reagent peptide with molecular hydrogen enriched with parahydrogen and a hydrogenation catalyst under conditions effective to form a hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of the disease in the subject and/or is a diagnosis of the disease in the subject.

In various embodiments, the present invention provides a method for diagnosing a disease in a subject, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of the disease in the subject and/or is a diagnosis of the disease in the subject.

In various embodiments, the present invention provides a method for diagnosing a disease in a subject, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of the disease in the subject and/or is a diagnosis of the disease in the subject.

In some embodiments, the image from the reference sample is from a control subject, wherein the control subject does not have the disease. In some embodiments, the image from the reference sample is a preexisting image from the subject. In some embodiments, the preexisting image corresponds to a baseline image. In some embodiments, the reference sample is from a healthy subject. In some embodiments, the reference sample is from a control subject, wherein the control subject does not have the disease. In some embodiments, the reference sample is obtained from the subject before the subject is treated for the disease. In some embodiments, the reference sample is from a subject that has been successfully treated for the disease. In some embodiments the reference sample is a sample obtained from the subject at an earlier point in time. In some embodiments, the method further comprises selecting or prescribing a treatment for the subject based on the diagnosis. In some embodiments, the method further comprises treating the subject based on the diagnosis. In some embodiments the method further comprises administering a treatment to the subject based on the diagnosis. In some embodiments the method further comprises providing a treatment to the subject based on the diagnosis. In some embodiments, the method further comprises referring the subject to a specialist based on the diagnosis. In some embodiments, the image from the reference sample is a magnetic resonance image.

Assessing and/or Determining the Risk of Developing a Disease

In various embodiments, the present invention provides a method for assessing and/or determining risk of developing a disease in a subject, comprising: providing a reagent peptide comprising at least one dehydroalanine residue and one or more other amino acid residues; contacting the reagent peptide with molecular hydrogen enriched with parahydrogen and a hydrogenation catalyst under conditions effective to form a hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of and/or is a determination of an increased risk of the subject developing the disease.

In some embodiments, the method further comprises selecting or prescribing a treatment for the subject based on the determination. In some embodiments, the method further comprises treating the subject based on the determination. In some embodiments the method further comprises administering a treatment to the subject based on the determination. In some embodiments the method further comprises providing a treatment to the subject based on the determination. In some embodiments, the method further comprises referring the subject to a specialist based on the determination.

In various embodiments, the present invention provides a method for assessing and/or determining risk of developing a disease in a subject, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative and/or is a determination of an increased risk of the subject developing the disease.

In various embodiments, the present invention provides a method for assessing and/or determining risk of developing a disease in a subject, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of and/or is a determination of an increased risk of the subject developing the disease.

In some embodiments, the image from the reference sample is from a control subject, wherein the control subject does not have the disease. In some embodiments, the image from the reference sample is a preexisting image from the subject. In some embodiments, the preexisting image corresponds to a baseline image. In some embodiments, the reference sample is from a healthy subject. In some embodiments, the reference sample is from a control subject, wherein the control subject does not have the disease. In some embodiments, the reference sample is obtained from the subject before the subject is treated for the disease. In some embodiments, the reference sample is from a subject that has been successfully treated for the disease. In some embodiments the reference sample is a sample obtained from the subject at an earlier point in time. In some embodiments, the method further comprises selecting or prescribing a treatment for the subject based on the determination. In some embodiments, the method further comprises treating the subject based on the determination. In some embodiments the method further comprises administering a treatment to the subject based on the determination. In some embodiments the method further comprises providing a treatment to the subject based on the determination. In some embodiments, the method further comprises referring the subject to a specialist based on the determination. In some embodiments the treatment is a preventative treatment. In some embodiments, the image from the reference sample is a magnetic resonance image.

Methods for Identifying and/or Detecting Disease

In various embodiments, the present invention provides a method for identifying and/or detecting a disease in a subject, comprising: providing a reagent peptide comprising at least one dehydroalanine residue and one or more other amino acid residues; contacting the reagent peptide with molecular hydrogen enriched with parahydrogen and a hydrogenation catalyst under conditions effective to form a hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is an identification and/or detection of the disease in the subject.

In various embodiments, the present invention provides a method for identifying and/or detecting a disease in a subject, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is an identification and/or detection of the disease in the subject.

In various embodiments, the present invention provides a method for identifying and/or detecting a disease in a subject, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is an identification and/or detection of the disease in the subject.

In some embodiments, the image from the reference sample is from a control subject, wherein the control subject does not have the disease. In some embodiments, the image from the reference sample is a preexisting image from the subject. In some embodiments, the preexisting image corresponds to a baseline image. In some embodiments, the reference sample is from a healthy subject. In some embodiments, the reference sample is from a control subject, wherein the control subject does not have the disease. In some embodiments, the reference sample is obtained from the subject before the subject is treated for the disease. In some embodiments, the reference sample is from a subject that has been successfully treated for the disease. In some embodiments the reference sample is a sample obtained from the subject at an earlier point in time. In some embodiments, the method further comprises selecting or prescribing a treatment for the subject based on the identification and/or detection of the disease. In some embodiments, the method further comprises treating the subject based on the identification and/or detection of the disease. In some embodiments the method further comprises administering a treatment to the subject based on the identification and/or detection of the disease. In some embodiments the method further comprises providing a treatment to the subject based on the identification and/or detection of the disease. In some embodiments, the method further comprises referring the subject to a specialist based on the identification and/or detection of the disease. In some embodiments, the image from the reference sample is a magnetic resonance image.

Prognostic Methods

In various embodiments, the present invention provides a method for prognosing a disease in a subject, comprising: providing a reagent peptide comprising at least one dehydroalanine residue and one or more other amino acid residues; contacting the reagent peptide with molecular hydrogen enriched with parahydrogen and a hydrogenation catalyst under conditions effective to form a hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is a prognosis of the disease in the subject.

In various embodiments, the present invention provides a method for prognosing a disease in a subject, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is a prognosis of the disease in the subject.

In various embodiments, the present invention provides a method for prognosing a disease in a subject, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is a prognosis of the disease in the subject.

In some embodiments, the image from the reference sample is from a control subject, wherein the control subject does not have the disease. In some embodiments, the image from the reference sample is a preexisting image from the subject. In some embodiments, the preexisting image corresponds to a baseline image. In some embodiments, the reference sample is from a healthy subject. In some embodiments, the reference sample is from a control subject, wherein the control subject does not have the disease. In some embodiments, the reference sample is obtained from the subject before the subject is treated for the disease. In some embodiments, the reference sample is from a subject that has been successfully treated for the disease. In some embodiments the reference sample is a sample obtained from the subject at an earlier point in time. In some embodiments, the method further comprises selecting or prescribing a treatment for the subject based on the prognosis. In some embodiments, the method further comprises treating the subject based on the prognosis. In some embodiments the method further comprises administering a treatment to the subject based on the prognosis. In some embodiments the method further comprises providing a treatment to the subject based on the prognosis. In some embodiments, the method further comprises referring the subject to a specialist based on the prognosis. In some embodiments, the image from the reference sample is a magnetic resonance image.

Methods for Determining Progression of a Disease

In various embodiments, the present invention provides a method for determining progression of a disease in a subject, comprising: providing a reagent peptide comprising at least one dehydroalanine residue and one or more other amino acid residues; contacting the reagent peptide with molecular hydrogen enriched with parahydrogen and a hydrogenation catalyst under conditions effective to form a hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of and/or is a determination of progression of the disease in the subject.

In various embodiments, the present invention provides a method for determining progression of a disease in a subject, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of and/or is a determination of progression of the disease in the subject.

In various embodiments, the present invention provides a method for determining progression of a disease in a subject, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of and/or is a determination of progression of the disease in the subject.

In some embodiments, the image from the reference sample is from a control subject, wherein the control subject does not have the disease. In some embodiments, the image from the reference sample is a preexisting image from the subject. In some embodiments, the preexisting image corresponds to a baseline image. In some embodiments, the reference sample is from a healthy subject. In some embodiments, the reference sample is from a control subject, wherein the control subject does not have the disease. In some embodiments, the reference sample is obtained from the subject before the subject is treated for the disease. In some embodiments, the reference sample is from a subject that has been successfully treated for the disease. In some embodiments the reference sample is a sample obtained from the subject at an earlier point in time. In some embodiments, the method further comprises selecting or prescribing a treatment for the subject based on the determination. In some embodiments, the method further comprises treating the subject based on the determination. In some embodiments the method further comprises administering a treatment to the subject based on the determination. In some embodiments the method further comprises providing a treatment to the subject based on the determination. In some embodiments, the method further comprises referring the subject to a specialist based on the determination. In some embodiments, the image from the reference sample is a magnetic resonance image.

Methods of Treating a Disease

In various embodiments, the present invention provides a method for treating a subject in need thereof, comprising: providing a reagent peptide comprising at least one dehydroalanine residue and one or more other amino acid residues; contacting the reagent peptide with molecular hydrogen enriched with parahydrogen and a hydrogenation catalyst under conditions effective to form a hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is a prognosis and/or a diagnosis of a disease in the subject; and treating the subject and/or selecting a treatment for and/or providing a treatment to the subject based on the prognosis and/or diagnosis of the disease in the subject. In some embodiments the treatment is a preventative treatment.

In various embodiments, the present invention provides a method for treating a subject in need thereof, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is a prognosis and/or a diagnosis of a disease in the subject; and treating the subject and/or selecting a treatment for and/or providing a treatment to the subject based on the prognosis and/or diagnosis of the disease in the subject. In some embodiments the treatment is a preventative treatment.

In various embodiments, the present invention provides a method for treating a subject in need thereof, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is a prognosis and/or a diagnosis of a disease in the subject; and treating the subject and/or selecting a treatment for and/or providing a treatment to the subject based on the prognosis and/or diagnosis of the disease in the subject. In some embodiments the treatment is a preventative treatment.

In various embodiments, the present invention provides a method for treating a subject for a disease, comprising: making an assessment of the subject based on a magnetic resonance image of the subject, wherein the assessment is a detection of the disease; and treating the subject based on the assessment.

In various embodiments, the present invention provides a method for assessing the efficacy of the treatment comprising: comparing the magnetic resonance image from the subject to an image from a reference sample, wherein a change in the image from the subject relative to the image from the reference sample is indicative of the efficacy of the treatment.

In some embodiments, the image from the reference sample is from a control subject, wherein the control subject does not have the disease. In some embodiments, the image from the reference sample is a preexisting image from the subject. In some embodiments, the preexisting image corresponds to a baseline image. In some embodiments, the reference sample is from a healthy subject. In some embodiments, the reference sample is from a control subject, wherein the control subject does not have the disease. In some embodiments, the reference sample is obtained from the subject before the subject is treated for the disease. In some embodiments, the reference sample is from a subject that has been successfully treated for the disease. In some embodiments the reference sample is a sample obtained from the subject at an earlier point in time. In some embodiments, the image from the reference sample is a magnetic resonance image.

Methods for Selecting a Subject for Treatment

In various embodiments, the present invention provides a method for selecting a subject for a treatment or therapy for a disease, comprising: providing a reagent peptide comprising at least one dehydroalanine residue and one or more other amino acid residues; contacting the reagent peptide with molecular hydrogen enriched with parahydrogen and a hydrogenation catalyst under conditions effective to form a hydrogenated peptide; subjecting the para-hydrogenated peptide to a polarization transfer sequence to obtain a hyperpolarized peptide; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of the disease in the subject and/or is a diagnosis of the disease in the subject; and selecting the subject for the treatment or therapy for the disease.

In various embodiments, the present invention provides a method for selecting a subject for a treatment or therapy for a disease, comprising: providing a hyperpolarized peptide, wherein the hyperpolarized peptide comprises at least one hyperpolarized alanine residue and one or more other amino acid residues; administering the hyperpolarized peptide to the subject; detecting magnetic resonance signals of the hyperpolarized peptide from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of the disease in the subject and/or is a diagnosis of the disease in the subject; and selecting the subject for the treatment or therapy for the disease.

In various embodiments, the present invention provides a method for selecting a subject for a treatment or therapy for a disease, comprising: providing a hyperpolarized compound, wherein the hyperpolarized compound comprises at least one hyperpolarized alanine residue; administering the hyperpolarized compound to the subject; detecting magnetic resonance signals of the hyperpolarized compound from the subject; generating one or more magnetic resonance images of the subject from the detected magnetic resonance signals; comparing the magnetic resonance images from the subject to an image from a reference sample, wherein a change in the magnetic resonance image from the subject relative to the image from the reference sample is indicative of the disease in the subject and/or is a diagnosis of the disease in the subject; and selecting the subject for the treatment or therapy for the disease.

In some embodiments, the image from the reference sample is from a control subject, wherein the control subject does not have the disease. In some embodiments, the image from the reference sample is a preexisting image from the subject. In some embodiments, the preexisting image corresponds to a baseline image. In some embodiments, the reference sample is from a healthy subject. In some embodiments, the reference sample is from a control subject, wherein the control subject does not have the disease. In some embodiments, the reference sample is obtained from the subject before the subject is treated for the disease. In some embodiments, the reference sample is from a subject that has been successfully treated for the disease. In some embodiments the reference sample is a sample obtained from the subject at an earlier point in time. In some embodiments, the image from the reference sample is a magnetic resonance image.

Specialists

In some embodiments the specialist is a cardiovascular physician, a heart failure physician, a cardiologist, a vascular physician, an electrophysiologist, a cardiovascular surgeon, an interventional physician, an imaging physician, a preventive cardiologist, a cardiothoracic surgeon, a vascular surgeon, an oncologist, a neurologist, a neurosurgeon, a radiologist, a urologist, a gastroenterologist, a dermatologist, an ophthalmologist, a psychiatrist, a medical oncologist, a clinical oncologist, or an endocrinologist.

Kits

The exact nature of the components configured in the inventive kit depends on its intended purpose. In one embodiment, the kit is configured particularly for human subjects. In further embodiments, the kit is configured for research and/or veterinary applications, treating subjects such as, but not limited to, farm animals, domestic animals, and laboratory animals (e.g., mouse or mice).

The materials or components assembled in the kit can be provided to the practitioner stored in any convenient and suitable ways that preserve their operability and utility. For example, the components can be dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit such as inventive peptides and compounds, and compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) a reagent peptide comprising at least one dehydroalanine residue and one or more other amino acid residues; and (b) and instructions for using the kit. In some embodiments, the kit further comprises molecular hydrogen enriched with parahydrogen. In some embodiments, the kit further comprises a hydrogenation catalyst.

In various embodiments, the present invention provides a kit for preparing a para-hydrogenated peptide, the kit comprising: (a) a reagent peptide comprising at least one dehydroalanine residue and one or more other amino acid residues; (b) molecular hydrogen enriched with parahydrogen; (c) a hydrogenation catalyst; and (d) instructions for preparing a para-hydrogenated peptide.

In various embodiments, the present invention provides a kit for preparing a hyperpolarized peptide, the kit comprising: (a) a reagent peptide comprising at least one dehydroalanine residue and one or more other amino acid residues; (b) molecular hydrogen enriched with parahydrogen; (c) a hydrogenation catalyst; (d) instructions for preparing a para-hydrogenated peptide; and (e) instructions for preparing a hyperpolarized peptide.

In various embodiments, the present invention provides a kit for imaging a subject, the kit comprising: (a) a reagent peptide comprising at least one dehydroalanine residue and one or more other amino acid residues; (b) molecular hydrogen enriched with parahydrogen; (c) a hydrogenation catalyst; (d) reagents and instructions for sample processing and preparation; and (e) instructions for using the kit for imaging the subject or to provide images of the subject. In some embodiments, the imaging the subject comprises magnetic resonance imaging (MRI) of the subject. In some embodiments, the images of the subject are magnetic resonance images.

In various embodiments, the present invention provides a kit for diagnosing a disease in a subject, the kit comprising: (a) a reagent peptide comprising at least one dehydroalanine residue and one or more other amino acid residues; (b) reagents and instructions for sample processing and preparation; and (c) instructions for using the kit to provide a diagnosis of the disease in the subject. In some embodiments, the kit further comprises molecular hydrogen enriched with parahydrogen. In some embodiments, the kit further comprises a hydrogenation catalyst.

In various embodiments, the present invention provides a kit for prognosing a disease in a subject, the kit comprising: (a) a reagent peptide comprising at least one dehydroalanine residue and one or more other amino acid residues; (b) reagents and instructions for sample processing and preparation; and (c) instructions for using the kit to provide a prognosis of the disease in the subject. In some embodiments, the kit further comprises molecular hydrogen enriched with parahydrogen. In some embodiments, the kit further comprises a hydrogenation catalyst.

In various embodiments, the present invention provides a kit for identifying a disease in a subject, the kit comprising: (a) a reagent peptide comprising at least one dehydroalanine residue and one or more other amino acid residues; (b) reagents and instructions for sample processing and preparation; and (c) instructions for using the kit to identify the disease in the subject. In some embodiments, the kit further comprises molecular hydrogen enriched with parahydrogen. In some embodiments, the kit further comprises a hydrogenation catalyst.

In various embodiments, the present invention provides a kit for detecting a disease in a subject, the kit comprising: (a) a reagent peptide comprising at least one dehydroalanine residue and one or more other amino acid residues; (b) reagents and instructions for sample processing and preparation; and (c) instructions for using the kit to detect the disease in the subject. In some embodiments, the kit further comprises molecular hydrogen enriched with parahydrogen. In some embodiments, the kit further comprises a hydrogenation catalyst.

In various embodiments, the present invention provides a kit for determining progression of a disease in a subject, the kit comprising: (a) a reagent peptide comprising at least one dehydroalanine residue and one or more other amino acid residues; (b) reagents and instructions for sample processing and preparation; and (c) instructions for using the kit to determine the progression of the disease in the subject. In some embodiments, the kit further comprises molecular hydrogen enriched with parahydrogen. In some embodiments, the kit further comprises a hydrogenation catalyst.

Some embodiments of the present invention can be defined as any of the following numbered paragraphs:

1. A hyperpolarized peptide, comprising: at least one hyperpolarized alanine residue and one or more other amino acid residues.
2. The hyperpolarized peptide of paragraph 1, wherein the one or more other amino acid residues is any natural amino acid or unnatural amino acid.
3. The hyperpolarized peptide of paragraph 1, wherein the one or more other amino acid residues is any natural amino acid.
4. The hyperpolarized peptide of paragraph 1, wherein the at least one hyperpolarized alanine residue comprises a non-hydrogen non-zero nuclear spin nucleus.
5. The hyperpolarized peptide of paragraph 4, wherein the non-hydrogen non-zero nuclear spin nucleus is ¹³C.
6. The hyperpolarized peptide of paragraph 5, wherein the ¹³C is present at a level according to its natural isotopic abundance.
7. The hyperpolarized peptide of paragraph 5, wherein the ¹³C is present at a level greater than its natural isotopic abundance.
8. The hyperpolarized peptide of paragraph 1, wherein one or more hydrogen atoms is replaced with a deuterium atom.
9. A method for preparing a hyperpolarized peptide, comprising:
a. providing a reagent peptide comprising at least one dehydroalanine residue and one or more other amino acid residues;
b. contacting the reagent peptide with molecular hydrogen enriched with para-hydrogen and a hydrogenation catalyst under conditions effective to form a para-hydrogenated peptide; and
c. subjecting the para-hydrogenated peptide to a polarization transfer sequence to obtain the hyperpolarized peptide.
   10. The method of paragraph 9, wherein the one or more other amino acid residues is any natural amino acid or unnatural amino acid.
   11. A method of imaging a subject, comprising:

a. administering a hyperpolarized peptide of paragraph 1 to the subject; and

b. imaging the subject after administering the hyperpolarized peptide to the subject.

12. The method of paragraph 11, wherein the imaging the subject comprises magnetic resonance imaging (MRI) of the subject.
13. The method of paragraph 11, further comprising diagnosing the subject as having a disease.
14. The method of paragraph 11, further comprising prognosing the subject as being likely to develop a disease.
15. The method of paragraph 11, further comprising prognosing the subject as having a higher probability of developing a disease as compared to a control subject, wherein the control subject does not have the disease.
16. A method for obtaining one or more magnetic resonance images of a subject, comprising:
a. providing a reagent peptide comprising at least one dehydroalanine residue and one or more other amino acid residues;
b. contacting the reagent peptide with molecular hydrogen enriched with para-hydrogen and a hydrogenation catalyst under conditions effective to form a para-hydrogenated peptide;
c. subjecting the para-hydrogenated peptide to a polarization transfer sequence to obtain a hyperpolarized peptide;
d. administering the hyperpolarized peptide to the subject;
e. detecting magnetic resonance signals of the hyperpolarized peptide from the subject; and
f. generating one or more magnetic resonance images of the subject from the detected magnetic resonance signals.
   17. A method for detecting a disease in a subject, comprising: comparing the magnetic resonance image of the subject from paragraph 16 to an image from a reference sample, wherein a change in the magnetic resonance image of the subject from paragraph 16 relative to the image from the reference sample is indicative of the disease in the subject.
   18. The method of paragraph 17, wherein the reference sample is obtained from a control subject, wherein the control subject does not have the disease.
   19. The method of paragraph 17, wherein the image from the reference sample is a preexisting image from the subject.
   20. The method of paragraph 19, wherein the preexisting image corresponds to a baseline image.
   21. A method for treating a subject for a disease, the method comprising:
a. making an assessment of the subject based on the magnetic resonance image of the subject of paragraph 16, wherein the assessment is a detection of the disease; and
b. treating the subject based on the assessment.
   22. A method for assessing the efficacy of the treatment of paragraph 21, comprising: comparing the magnetic resonance image from the subject to an image from a reference sample, wherein a change in the image from the subject relative to the image from the reference sample is indicative of the efficacy of the treatment.
   23. The method of paragraph 22, wherein the reference sample is obtained from a control subject, wherein the control subject does not have the disease.
   24. The method of paragraph 22, wherein the reference sample is obtained from the subject before the subject is treated for the disease.
   25. The method of paragraph 22, wherein the reference sample is from a subject that has been successfully treated for the disease.
   26. The method of paragraph 17 or paragraph 22, wherein the image from the reference sample is a magnetic resonance image.

### EXAMPLES

The invention is further illustrated by the following examples which are intended to be purely exemplary of the invention, and which should not be construed as limiting the invention in any way. The following examples are illustrative only, and are not intended to limit, in any manner, any of the aspects described herein. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

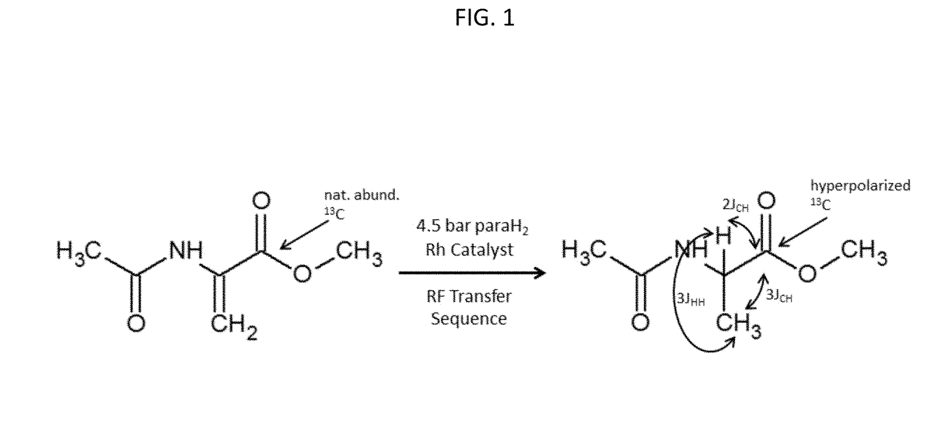

Our objective was to test a model system of polarization that can be incorporated into peptide sequence. Dehydroalanine is an unnatural amino acid with a methylene group instead of the normal methyl side chain for alanine. Several publications laid out the framework on synthesizing peptides containing this reactive amino acid side chain double bond. (Okeley N M, Zhu Y, van der Donk W A. Facile Chemoselective Synthesis of Dehydroalanine-Containing Peptides†. Organic Letters. 2000; 2(23):3603-6). The double bond of dehydroalanine is ideally positioned for PHIP to generate a polarized ¹³C carboxyl nucleus with the resulting product an alanine amino acid residue. We utilized a purchased chemical equivalent resembling dehydroalanine in a peptide chain with the terminal carboxyl and amine protected by functional groups (FIG. 1). A 70 mL 2 mM rhodium catalyst (Gridnev I D, Higashi N, Asakura K, Imamoto T. Mechanism of Asymmetric Hydrogenation Catalyzed by a Rhodium Complex of (S,S)-1,2 Bis(tertbutylmethylphosphino) ethane. Dihydride Mechanism of Asymmetric Hydrogenation. Journal of the American Chemical Society. 2000; 122(30):7183-94) with 24 mM methyl 2-acetamidoacrylate (MMA) deuterium oxide solution was prepared and stored in an N₂ atmosphere. 4 mL aliquots of solution were injected into a 1.4 mT PHIP instrument. (Agraz J, Grunfeld A, Cunningham K, Li D, Wagner S. Improved PHIP polarization using a precision, low noise, voltage controlled current source. J Magn Reson. 2013; 235:77-84. Epub 2013 Aug. 31). Samples were heated to 50° Celsius, then reacted with 4.5 bar of >99% purity para-hydrogen for a 3 second hydrogenation with hydrogen decoupling (MLEV-16), followed by a Goldman RF transfer sequence. (Goldman M, Jóhannesson H. Conversion of a proton pair para order into 13C polarization by rf irradiation, for use in MRI. Comptes Rendus Physique. 2005; 6(4-5):575-81). The sequence was varied to validate the carbon-hydrogen coupling constants (2J_{CH} and 3J_{CH}) estimated from similar chemical structures. The 3J_{HH} scalar coupling (J) constant was kept at 7.47 Hz, the 3J_{HH} value was determined by proton spectroscopy. All measured samples were combined and lyophilized to dryness, then and dissolved in 2.5 mL of diH₂O and scanned (¹³C spectroscopy) for 17 hours with a repetition time of 50 seconds.

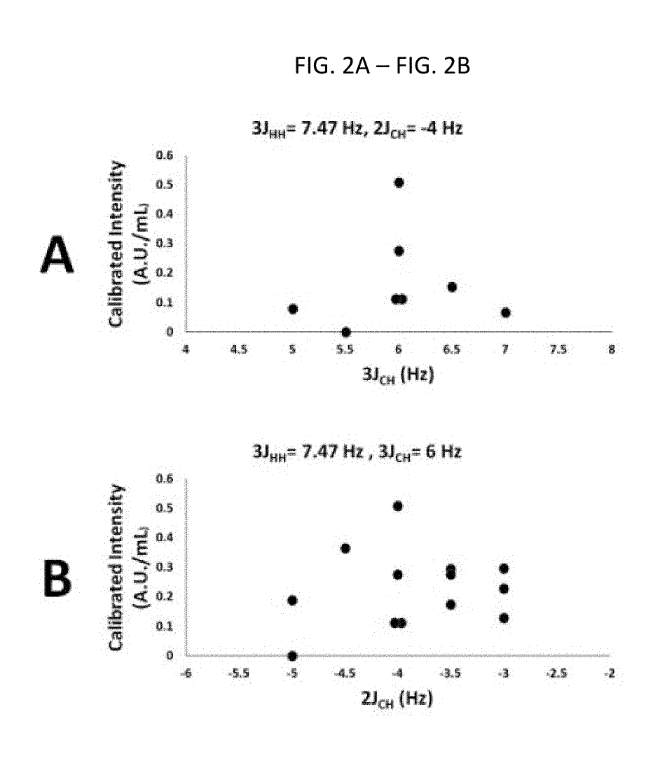

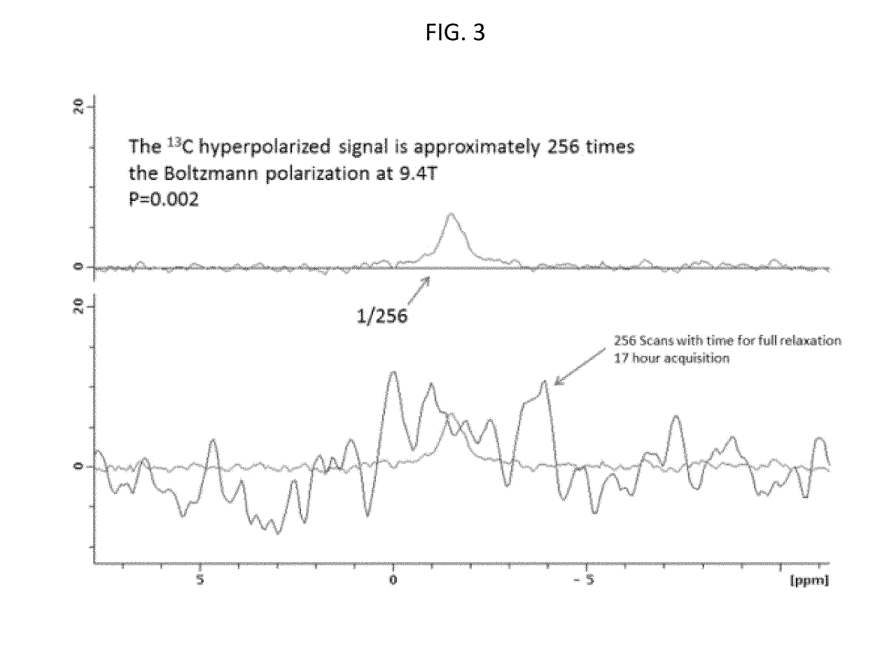

We obtained viable polarization and observed that the coupling constant accuracy was crucial for high polarization transfer to the carboxyl carbon. The polarization trend showed high variance as illustrated in FIG. 2A and FIG. 2B. The best set of scalar coupling constants reached 0.1±0.07% polarization. The maximum polarization from the best sequence was approximated to be 0.2% based on the Boltzmann thermal equilibrium signal (FIG. 3). ¹H spectroscopy clearly visualized the signal from the product and did not show any signal above the signal-to-noise for the MAA (not shown); therefore, we expect the reaction yield to be greater than 95% for the 3 second reaction time.

The detected polarization was achieved with a non-deuterated natural abundance ¹³C test molecule at low concentration with a single 90 degree pulse. This is a remarkable outcome since the intramolecular relaxation of the protons result in significant relaxation after the para-hydrogen molecule is added. The large variation in the acquired signal is likely a direct result of the fast relaxation combined with fluctuations in the chemical reaction time caused by varied timing of placing the sample. These timing variations impact the average pre-transfer relaxation time of the two hydrogens inserted onto the MAA molecule. The direct consequence of different completion times of the chemical reaction prior to transfer results in a different amount of relaxation of the inserted para-hydrogen protons. Our high product yield leads us to suspect that polarization can be improved with a lower catalyst concentration to slow down the chemical conversion for the reaction to be completed at the 3 second time point.

These initial polarization results indicate an expanded clinical application spectrum of hyperpolarized MRI, specifically with PHIP on account of its performance in aqueous solutions. Even without deuteration of nearby hydrogens we produced substantial ¹³C polarization.

To provide aspects of the present disclosure, embodiments may employ any number of programmable processing devices that execute software or stored instructions. Physical processors and/or machines employed by embodiments of the present disclosure for any processing or evaluation may include one or more networked (Internet, cloud, WAN, LAN, satellite, wired or wireless (RF, cellular, WiFi, Bluetooth, etc.)) or non-networked general purpose computer systems, microprocessors, filed programmable gate arrays (FPGAs), digital signal processors (DSPs), micro-controllers, smart devices (e.g., smart phones), computer tablets, handheld computers, and the like, programmed according to the teachings of the exemplary embodiments. In addition, the devices and subsystems of the exemplary embodiments can be implemented by the preparation of application-specific integrated circuits (ASICs) or by interconnecting an appropriate network of conventional component circuits. Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, database management software, and the like. Computer code devices of the exemplary embodiments can include any suitable interpretable or executable code mechanism, including but not limited to scripts, interpretable programs, dynamic link libraries (DLLs), Java classes and applets, complete executable programs, and the like. Moreover, processing capabilities may be distributed across multiple processors for better performance, reliability, cost, or other benefits.

Common forms of computer-readable media may include, for example, a floppy disk, a flexible disk, a hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read. Such storage media can also be employed to store other types of data, e.g., data organized in a database, for access, processing, and communication by the processing devices.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A hyperpolarized peptide, comprising: at least one hyperpolarized alanine residue and one or more other amino acid residues.

2. The hyperpolarized peptide of claim 1, wherein the one or more other amino acid residues is any natural amino acid or unnatural amino acid.

3. The hyperpolarized peptide of claim 1, wherein the one or more other amino acid residues is any natural amino acid.

4. The hyperpolarized peptide of claim 1, wherein the at least one hyperpolarized alanine residue comprises a non-hydrogen non-zero nuclear spin nucleus.

5. The hyperpolarized peptide of claim 4, wherein the non-hydrogen non-zero nuclear spin nucleus is ¹³C.

6. The hyperpolarized peptide of claim 5, wherein the ¹³C is present at a level according to its natural isotopic abundance.

7. The hyperpolarized peptide of claim 5, wherein the ¹³C is present at a level greater than its natural isotopic abundance.

8. The hyperpolarized peptide of claim 1, wherein one or more hydrogen atoms is replaced with a deuterium atom.

9. A method for preparing a hyperpolarized peptide, comprising:
providing a reagent peptide comprising at least one dehydroalanine residue and one or more other amino acid residues;
contacting the reagent peptide with molecular hydrogen enriched with para-hydrogen and a hydrogenation catalyst under conditions effective to form a para-hydrogenated peptide; and
subjecting the para-hydrogenated peptide to a polarization transfer sequence to obtain the hyperpolarized peptide.

10. The method of claim 9, wherein the one or more other amino acid residues is any natural amino acid or unnatural amino acid.

11. A method of imaging a subject, comprising:
administering a hyperpolarized peptide of claim 1 to the subject; and
imaging the subject after administering the hyperpolarized peptide to the subject.

12. The method of claim 11, wherein the imaging the subject comprises magnetic resonance imaging (MRI) of the subject.

13. The method of claim 11, further comprising diagnosing the subject as having a disease.

14. The method of claim 11, further comprising prognosing the subject as being likely to develop a disease.

15. The method of claim 11, further comprising prognosing the subject as having a higher probability of developing a disease as compared to a control subject, wherein the control subject does not have the disease.

16. A method for obtaining one or more magnetic resonance images of a subject, comprising:
providing a reagent peptide comprising at least one dehydroalanine residue and one or more other amino acid residues;
contacting the reagent peptide with molecular hydrogen enriched with para-hydrogen and a hydrogenation catalyst under conditions effective to form a para-hydrogenated peptide;
subjecting the para-hydrogenated peptide to a polarization transfer sequence to obtain a hyperpolarized peptide;
administering the hyperpolarized peptide to the subject;
detecting magnetic resonance signals of the hyperpolarized peptide from the subject; and
generating one or more magnetic resonance images of the subject from the detected magnetic resonance signals.

17. A method for detecting a disease in a subject, comprising: comparing the magnetic resonance image of the subject from claim 16 to an image from a reference sample, wherein a change in the magnetic resonance image of the subject from claim 16 relative to the image from the reference sample is indicative of the disease in the subject.

18. The method of claim 17, wherein the reference sample is obtained from a control subject, wherein the control subject does not have the disease.

19. The method of claim 17, wherein the image from the reference sample is a preexisting image from the subject.

20. The method of claim 19, wherein the preexisting image corresponds to a baseline image.

21. A method for treating a subject for a disease, the method comprising:
making an assessment of the subject based on the magnetic resonance image of the subject of claim 16, wherein the assessment is a detection of the disease; and
treating the subject based on the assessment.

22. A method for assessing the efficacy of the treatment of claim 21, comprising: comparing the magnetic resonance image from the subject to an image from a reference sample, wherein a change in the image from the subject relative to the image from the reference sample is indicative of the efficacy of the treatment.

23. The method of claim 22, wherein the reference sample is obtained from a control subject, wherein the control subject does not have the disease.

24. The method of claim 22, wherein the reference sample is obtained from the subject before the subject is treated for the disease.

25. The method of claim 22, wherein the reference sample is from a subject that has been successfully treated for the disease.

26. The method of claim 17, wherein the image from the reference sample is a magnetic resonance image.
